(19) United States
(12) Patent Application Publication
(10) Pub. No.: US 2010/0004271 A1
(43) Pub. Date: Jan. 7, 2010

(54) HETEROCYCLIC COMPOUNDS AS AGANIST FOR THE THYROID RECEPTOR

(75) Inventors: Ana Maria Garcia Collazo, Madrid (ES); Eva Kristina Koch, Huddinge (SE); Anton Joakim Lofstedt, Uppsala (SE); Aiping Cheng, Huddinge (SE); Thomas Fredrik Hansson, Huddinge (SE); Edouard Zamaratski, Huddinge (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: KARO BIO AB, Huddinge (SE)

(21) Appl. No.: 11/922,554

(22) PCT Filed: Jul. 4, 2006
(86) PCT No.: PCT/EP2006/006522

§ 371 Date: Sep. 4, 2009

(30) Foreign Application Priority Data
Jul. 4, 2005 (GB) 0513692.4

(51) Int. Cl. A61K 31/517; C07D 215/18; C07D 215/38; C07D 215/16; C07D 277/62; C07D 263/54; C07D 235/12; C07D 239/72; A61K 31/47; A61K 31/428; A61K 31/423; A61K 31/416; G01N 33/566; A61P 9/10

(52) U.S. Cl. 514/266.1; 546/180; 546/175; 546/178; 548/178; 548/217; 548/310.1; 544/283; 514/311; 514/367; 514/375; 514/394; 436/501

(57) ABSTRACT

The invention provides Compounds of formula (I) or pharmaceutically acceptable esters, amides, solvates or salts thereof, including salts of such esters or amides, and solvates of such esters, amides or salts, wherein R³, R⁴, G, Y, W and R⁵ are as defined in the specification. The invention also provides the use of such Compounds in the treatment or Prophylaxis of a condition associated with a disease or disorder associated with thyroid receptor activity.

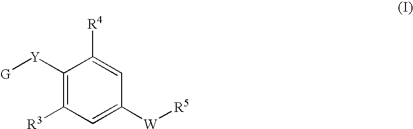

### FIELD OF THE INVENTION

The present invention relates to compounds which are agonists or partial agonists of the thyroid receptor and the use of such compounds for therapeutic purposes.

### BACKGROUND OF THE INVENTION

While the extensive role of thyroid hormones in regulating metabolism in humans is well recognized, the discovery and development of new specific drugs for improving the treatment of hyperthyroidism and hypothyroidism has been slow. This has also limited the development of thyroid agonists and antagonists for treatment of other important clinical indications, such as hypercholesterolemia, dyslipidemia, obesity, diabetes, atherosclerosis and cardiac diseases.

Thyroid hormones affect the metabolism of virtually every cell of the body. At normal levels, these hormones maintain body weight, metabolic rate, body temperature and mood, and influence blood levels of serum lipoproteins. Thus, in hypothyroidism there is weight gain, high levels of LDL cholesterol, and depression. In hyperthyroidism, these hormones lead to weight loss, hypermetabolism, lowering of serum LDL cholesterol levels, cardiac arrhythmias, heart failure, muscle weakness, bone loss in postmenopausal women, and anxiety.

Thyroid hormones are currently used primarily as replacement therapy for patients with hypothyroidism. Therapy with L-thyroxine returns metabolic functions to normal and can easily be monitored with routine serum measurements of levels of thyroid-stimulating hormone (TSH), thyroxine (3,5,3′,5′-tetraiodo-L-thyronine, or T₄) and triiodothyronine (3,5,3′-triiodo-L-thyronine, or T₃). However, replacement therapy, particularly in older individuals, may be restricted by certain detrimental effects from thyroid hormones.

In addition, some effects of thyroid hormones may be therapeutically useful in non-thyroid disorders if adverse effects can be minimized or eliminated. These potentially useful influences include for example, lowering of serum LDL levels, weight reduction, amelioration of depression and stimulation of bone formation. Prior attempts to utilize thyroid hormones pharmacologically to treat these disorders have been limited by manifestations of hyperthyroidism, and in particular by cardiovascular toxicity.

Furthermore, useful thyroid agonist drugs should minimize the potential for undesired consequences due to locally induced hypothyroidism, i.e. sub-normal levels of thyroid hormone activity in certain tissues or organs. This can arise because increased circulating thyroid hormone agonist concentrations may cause the pituitary to suppress the secretion of thyroid stimulating hormone (TSH), thereby reducing thyroid hormone synthesis by the thyroid gland (negative feedback control). Since endogenous thyroid hormone levels are reduced, localized hypothyroidism can result wherever the administered thyroid agonist drug fails to compensate for the reduction in endogenous hormone levels in specific tissues.

Development of specific and selective thyroid hormone receptor ligands, particularly agonists of the thyroid hormone receptor, is expected to lead to specific therapies for these common disorders, while avoiding the cardiovascular and other toxicity of native thyroid hormones. Tissue-selective thyroid hormone agonists may be obtained by selective tissue uptake or extrusion, topical or local delivery, targeting to cells through other ligands attached to the agonist and targeting receptor subtypes. Tissue selectivity can also be achieved by selective regulation of thyroid hormone responsive genes in a tissue specific manner.

Accordingly, the compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

### SUMMARY OF THE INVENTION

The present invention provides a compound of formula (I) or a pharmaceutically acceptable ester, amide, solvate or salt thereof, including a salt of such an ester or amide, and a solvate of such an ester, amide or salt,

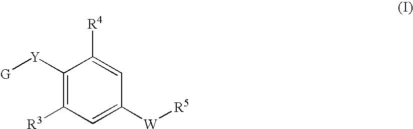

wherein:
G is a group selected from:

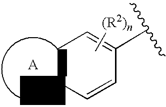

N is a sp² nitrogen with a non-bonded electron pair in an sp² orbital;
The ring A is an aromatic or a non-aromatic five-membered or six-membered ring optionally comprising one or more further heteroatoms independently selected from oxygen, sulfur, sp² nitrogen, and —N(R¹⁰)—, the carbon atoms of ring A optionally being substituted with one or more groups R¹;
Each R¹⁰ is independently selected from —(CH₂)ₚ—S—R^{b}, —(CH₂)ₚ—SO₂—R^{b}, —(CH₂)ₚ—NH—SO₂—R^{b}, —(CH₂)ₚ—SO₂—NH—R^{b}, —(CH₂)ₚ—NH—CO—R^{b}, —(CH₂)ₚ—CO—NH—R^{b}, C₁₋₁₂ alkyl, C₂₋₁₂ alkenyl, C₂₋₁₂ alkynyl, C₃₋₈ cycloalkyl, C₃₋₈ cycloalkyl-C₁₋₃ alkyl, phenyl, benzyl and C₃₋₇heterocyclyl, said alkyl, alkenyl or alkynyl groups or portions of groups optionally being substituted with 1, 2 or 3 groups, each independently selected from halogen, hydroxy, N(R^{a})₂, phenyl, C₁₋₄ alkoxy, haloC₁₋₄ alkoxy, dihaloC₁₋₄ alkoxy, and trihaloC₁₋₄ alkoxy; said cycloalkyl, phenyl, benzyl or heterocyclyl groups or portions of groups optionally being substituted with 1, 2 or 3 groups independently selected from halogen, hydroxy, N(R^{a})₂, C₁₋₄ alkyl, C₂₋₄ alkenyl, C₂₋₄ alkynyl, haloC₁₋₄alkyl, dihaloC₁₋₄alkyl, trihaloC₁₋₄alkyl, methoxy, halomethoxy, dihalomethoxy, and trihalomethoxy;
p is 1 or 2;
each R^{a} is independently selected from a hydrogen atom and a C₁₋₄ alkyl group optionally substituted with 1, 2 or 3 groups independently selected from halogen, methoxy, halomethoxy, dihalomethoxy and trihalomethoxy;
each R^{b} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy and trihalomethoxy;
Each R¹ is independently selected from hydrogen, hydroxy, halogen, N(R^{a})₂, —(CH₂)ₘ—S—R^{b}, —(CH₂)ₘ—SO₂—R^{b}, —(CH₂)ₘ—NH—SO₂—R^{b}, —(CH₂)ₘ—SO₂—NH—R^{b}, —(CH₂)ₘ—NH—CO—R^{b}, —(CH₂)ₘCO—NH—R^{b}, C₁₋₁₂ alkyl, C₂₋₁₂ alkenyl, C₂₋₁₂ alkynyl, C₃₋₈ cycloalkyl, C₃₋₈cycloalkyl-C₁₋₃ alkyl, phenyl, benzyl and C₃₋₇heterocyclyl, said alkyl, alkenyl or alkynyl groups or portions of groups optionally being substituted with 1, 2 or 3 groups each independently selected from halogen, hydroxy, N(R^{a})₂, phenyl, C₁₋₄ alkoxy, haloC₁₋₄ alkoxy, dihaloC₁₋₄ alkoxy, and trihaloC₁₋₄ alkoxy; said cycloalkyl, phenyl, benzyl or heterocyclyl groups or portions of groups optionally being substituted with 1, 2 or 3 groups independently selected from halogen, hydroxy, N(R^{a})₂, C₁₋₄ alkyl, C₂₋₄ alkenyl, C₂₋₄ alkynyl, haloC₁₋₄alkyl, dihaloC₁₋₄alkyl, trihaloC₁₋₄alkyl, methoxy, halomethoxy, dihalomethoxy, and trihalomethoxy;
m is 0, 1 or 2;
Each R² is independently selected from halogen, mercapto, cyano, C₁₋₄ alkoxy, C₁₋₄ alkyl, C₂₋₄ alkenyl, C₂₋₄ alkynyl and N(R^{a})₂, said alkyl, alkenyl, alkynyl or alkoxy groups optionally being substituted with 1, 2 or 3 groups selected from halogen, hydroxy, C₁₋₄ alkoxy, C₁₋₄ alkylthio, haloC₁₋₄ alkoxy, dihaloC₁₋₄ alkoxy, and trihaloC₁₋₄ alkoxy;
n is 0, 1 or 2;
Y is selected from oxygen, methylene, sulphur, SO, SO₂ and —N(R^{a})—;
R³ and R⁴ are independently selected from halogen, cyano, C₁₋₄ alkyl, C₂₋₄ alkenyl, C₂₋₄ alkynyl, haloC₁₋₄ alkyl, dihaloC₁₋₄ alkyl, trihaloC₁₋₄ alkyl, C₁₋₄ alkoxy, haloC₁₋₄ alkoxy, dihaloC₁₋₄ alkoxy, trihaloC₁₋₄ alkoxy, methylthio, halomethylthio, dihalomethylthio and trihalomethylthio;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NH(CO)—C₀₋₃ alkylene and C₁₋₃ alkyleneC(O)NH—C₁₋₃ alkylene, said alkylene, alkenylene or alkynylene groups or portions of groups optionally being substituted with 1 or 2 groups selected from hydroxy, mercapto, amino, halogen, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
R^{c} is selected from hydrogen, C₁₋₄ alkyl, C₂₋₄ alkenyl, C₂₋₄ alkynyl, C₁₋₄ alkoxy, haloC₁₋₄ alkyl, dihaloC₁₋₄ alkyl, trihaloC₁₋₄ alkyl, haloC₁₋₄ alkoxy, dihaloC₁₋₄ alkoxy, and trihaloC₁₋₄ alkoxy;
R⁵ is selected from —CO₂R^{d}, —PO(OR^{d})₂, —PO(OR^{c})NH₂, —SO₂OR^{d}, —COCO₂R^{d}, CONR^{d}OR^{d}, —SO₂NHR^{d}, —NHSO₂R^{d}, —CONHSO₂R^{d}, and —SO₂NHCOR^{d}; and
each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₋₄ alkyl.

Compounds of the invention have surprisingly been found to be ligands of the thyroid receptor, in particular agonists or partial agonists of the thyroid receptor. The compounds accordingly have use in the treatment or prophylaxis of conditions associated with thyroid receptor activity.

### DETAILED DESCRIPTION OF THE INVENTION

The compounds of formula (I) may contain chiral (asymmetric) centres or the molecule as a whole may be chiral. The individual stereoisomers (enantiomers and diastereoisomers) and mixtures of these are within the scope of the present invention.

The present invention provides compounds of formula (I) that are thyroid receptor ligands having a hydrogen bond acceptor in the prime ring while lacking a hydrogen bond donor.

Preferably G is a group selected from:

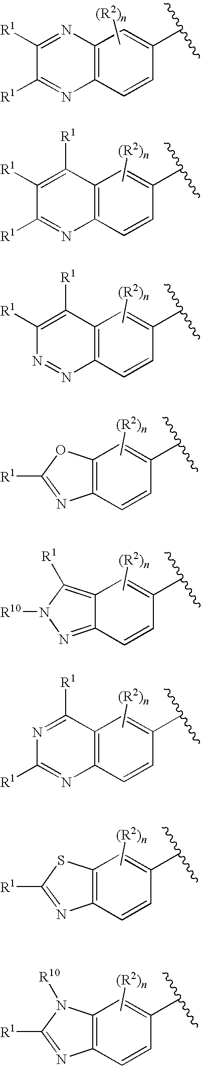

In one embodiment, G is a group selected from:

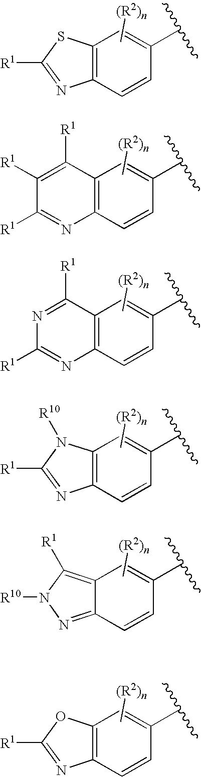

In another embodiment, G is a group selected from:

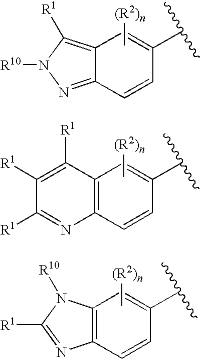

Preferably, R¹⁰ is selected from —(CH₂)ₚ—S—R^{b}, —(CH₂)ₚ—SO₂—R^{b}, —(CH₂)ₚ—NH—SO₂—R^{b}, —(CH₂)ₚ—SO₂—NH—R^{b}, —(CH₂)ₚ—NH—CO—R_{b}, —(CH₂)ₚ—CO—NH—R^{b}, C₁₋₈ alkyl, C₃₋₆ cycloalkyl, C₃₋₆ cycloalkyl-C₁₋₃ alkyl, phenyl, benzyl and C₃₋₇ heterocyclyl with optional substitution of those groups as described above. More preferably, R¹⁰ is selected from C₁₋₅ alkyl, phenyl and C₃₋₇ heterocyclyl with optional substitution of those groups as described above. Preferred substituents for said alkyl groups or portions of groups include groups independently selected from halogen, hydroxy, N(R^{a})₂, phenyl, methoxy, halomethoxy, dihalomethoxy, and trihalomethoxy. Accordingly, examples of preferred R¹⁰ groups include fluoroC₁₋₁₂alkyl, difluoroC₁₋₁₂ alkyl, and trifluoroC₁₋₁₂ alkyl. Preferred substituents for said cycloalkyl, phenyl or heterocyclyl groups or portions of groups include groups independently selected from halogen, N(R^{a})₂, hydroxy, C₁₋₄ alkyl, haloC₁₋₄alkyl, dihaloC₁₋₄alkyl, trihaloC₁₋₄alkyl, methoxy, halomethoxy, dihalomethoxy, and trihalomethoxy.

In one embodiment, each R¹⁰ is independently selected from C₁₋₁₂ alkyl, C₂₋₁₂ alkenyl, C₂₋₁₂ alkynyl, C₃₋₈ cycloalkyl, C₃₋₈ cycloalkyl-C₁₋₃ alkyl, phenyl, and C₃₋₇heterocyclyl, said alkyl, alkenyl or alkynyl groups or portions of groups optionally being substituted with 1, 2 or 3 groups each independently selected from halogen, hydroxy, phenyl, C₁₋₄ alkoxy, haloC₁₋₄ alkoxy, dihaloC₁₋₄ alkoxy, and trihaloC₁₋₄ alkoxy; said cycloalkyl, phenyl or heterocyclyl groups or portions of groups optionally being substituted with 1, 2 or 3 groups independently selected from halogen, hydroxy, C₁₋₄ alkyl, C₂₋₄ alkenyl, C₂₋₄ alkynyl, methoxy, halomethoxy, dihalomethoxy, and trihalomethoxy;

Preferably, p is 1 or 2. More preferably p is 1.

Preferably, R^{a} is independently selected from hydrogen and C₁₋₄ alkyl with optional substitution of those groups as described above. Preferred substituents for said C₁₋₄ alkyl include halogen groups.

Preferably, R^{a} is selected from hydrogen, C₁₋₄ alkyl and C₁₋₄ alkyl substituted with 1 to 3 halogen groups.

Preferably, R^{b} is selected from hydrogen, C₁₋₄ alkyl, fluoromethyl, difluoromethyl, or trifluoromethyl, benzyl, C₃₋₇ heterocyclyl and phenyl with optional substitution of those groups as described above. Preferred substituents for said C₁₋₄ alkyl or phenyl groups include halogen, hydroxy, methoxy, halomethoxy, dihalomethoxy, and trihalomethoxy.

Preferably, R¹ is selected from hydrogen, hydroxy, halogen, —(CH₂)ₘ—S—R^{b}, —(CH₂)ₘ—SO₂—R^{b}, —(CH₂)ₘ—NH—SO₂—R^{b}, —(CH₂)ₘ—SO₂—NH—R^{b}, —(CH₂)ₘ—NH—CO—R^{b}, —(CH₂)ₘ—CO—NH—R^{b}, C₁₋₈alkyl, C₃₋₆cycloalkyl, C₃₋₆cycloalkyl-C₁₋₃ alkyl, phenyl and C₃₋₅ heterocyclyl with optional substitution of those groups as described above. More preferably, R¹ is selected from hydrogen, hydroxy, halogen, C₁₋₅alkyl, phenyl, benzyl and C₃₋₅ heterocyclyl with optional substitution of those groups as described above. Preferred substituents for said alkyl groups or portions of groups include groups independently selected from halogen, hydroxy, N(R^{a})₂, phenyl, methoxy, halomethoxy, dihalomethoxy, and trihalomethoxy. Accordingly, examples of preferred R¹ groups include fluoroC₁₋₁₂ alkyl, difluoroC₁₋₁₂ alkyl, and trifluoroC₁₋₁₂ alkyl. Preferred substituents for said cycloalkyl, phenyl or heterocyclyl groups or portions of groups include groups independently selected from halogen, hydroxy, N(R^{a})₂, C₁₋₄ alkyl, haloC₁₋₄alkyl, dihaloC₁₋₄alkyl, trihaloC₁₋₄alkyl, methoxy, halomethoxy, dihalomethoxy, and trihalomethoxy.

Preferably, each R¹ is independently selected from hydrogen, hydroxy, halogen, C₁₋₁₂ alkyl, C₂₋₁₂ alkenyl, C₂₋₁₂ alkynyl, C₃₋₈ cycloalkyl, C₃₋₈ cycloalkyl-C₁₋₃ alkyl, phenyl and C₃₋₇heterocyclyl, said alkyl, alkenyl or alkynyl groups or portions of groups optionally being substituted with 1, 2 or 3 groups each independently selected from halogen, hydroxy, phenyl, C₁₋₄ alkoxy, haloC₁₋₄ alkoxy, dihaloC₁₋₄ alkoxy, and trihaloC₁₋₄ alkoxy; said cycloalkyl, phenyl, benzyl or heterocyclyl groups or portions of groups optionally being substituted with 1, 2 or 3 groups independently selected from halogen, hydroxy, C₁₋₄ alkyl, C₂₋₄ alkenyl, C₂₋₄ alkynyl, methoxy, halomethoxy, dihalomethoxy, and trihalomethoxy;

Preferably m is 0, 1 or 2. More preferably m is 0 or 1.

Preferably, each R² is independently selected from halogen, mercapto, C₁₋₄ alkoxy, C₁₋₄ alkyl and N(R^{a})₂ with optional substitution of those groups as described above. More preferably, R² is selected from halogen, C₁₋₄ alkoxy and C₁₋₄ alkyl with optional substitution of those groups as described above. Preferred substituents for said alkyl or alkoxy groups or portions of groups include groups independently selected from halogen, hydroxy, C₁₋₄ alkylthio, methoxy, halomethoxy, dihalomethoxy, and trihalomethoxy.

Preferably n is 0 or 1. More preferably n is 0.

In one embodiment G is a group:

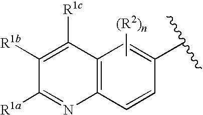

in which R^{1a} is selected from hydrogen, hydroxy, halogen, —(CH₂)ₘ—NH—SO₂—R^{b}, —(CH₂)ₘ—CO—NH—R^{b}, C₁₋₈alkyl, C₂₋₈alkenyl, C₂₋₈alkynyl, C₃₋₄cycloalkyl, C₃₋₆cycloalkyl-C₁₋₃ alkyl, phenyl and C₃₋₇ heterocyclyl with optional substitution of those groups as described above. More preferably, R^{1a} is selected from hydrogen, hydroxy, halogen, —(CH₂—NH—SO₂—R^{b}, —(CH₂—CO—NH—R^{b}, C₁₋₅alkyl, phenyl and C₃₋₇ heterocyclyl with optional substitution of those groups as described above. Preferred substituents for said alkyl groups or portions of groups include groups independently selected from halogen, hydroxy, phenyl, methoxy, halomethoxy, dihalomethoxy, and trihalomethoxy. Accordingly, examples of preferred R^{1a} groups include fluoroC₁₋₁₂ alkyl, difluoroC₁₋₂ alkyl, and trifluoroC₁₋₁₂alkyl. Preferred substituents for said cycloalkyl, phenyl or heterocyclyl groups or portions of groups include groups independently selected from halogen, hydroxy, C₁₋₄ alkyl, methoxy, halomethoxy, dihalomethoxy, and trihalomethoxy.

R^{1b} is selected from hydrogen, hydroxy, halogen, C₁₋₁₈alkyl, C₂₋₈ alkenyl, C2-g alkynyl, C₃₋₆cycloalkyl, C₃₋₄cycloalkyl-C₁₋₃ alkyl, phenyl and C₃₋₇ heterocyclyl with optional substitution of those groups as described above. More preferably, R^{1b} is selected from hydrogen, hydroxy, halogen, C₁₋₅ alkyl, phenyl and C₃₋₇ heterocyclyl with optional substitution of those groups as described above. Preferred substituents for said alkyl groups or portions of groups include groups independently selected from halogen, hydroxy, phenyl, methoxy, halomethoxy, dihalomethoxy, and trihalomethoxy. Accordingly, examples of preferred R^{1b} groups include fluoroC₁₋₁₂ alkyl, difluoroC₁₋₁₂ alkyl, and trifluoroC₁₋₁₂ alkyl. Preferred substituents for said cycloalkyl, phenyl or heterocyclyl groups or portions of groups include groups independently selected from halogen, hydroxy, C₁₋₄ alkyl, methoxy, halomethoxy, dihalomethoxy, and trihalomethoxy. In one embodiment, R^{1b} is hydrogen.

R^{1c} is selected from hydrogen, hydroxy, halogen, C₁₋₈alkyl, C₂₋₈ alkenyl, C₂₋₈ alkynyl, C₃₋₆cycloalkyl, C₃₋₆cycloalkyl-C₁₋₃ alkyl, phenyl and C₃₋₇ heterocyclyl with optional substitution of those groups as described above. More preferably, R^{1c} is selected from hydrogen, hydroxy, halogen, C₁₋₅ alkyl, phenyl and C₃₋₇ heterocyclyl with optional substitution of those groups as described above. Preferred substituents for said alkyl groups or portions of groups include groups independently selected from halogen, hydroxy, phenyl, methoxy, halomethoxy, dihalomethoxy, and trihalomethoxy. Accordingly, examples of preferred R^{1c} groups include fluoroC₁₋₁₂ alkyl, difluoroC₁₋₁₂ alkyl, and trifluoroC₁₋₁₂ alkyl. Preferred substituents for said cycloalkyl, phenyl or heterocyclyl groups or portions of groups include groups independently selected from halogen, hydroxy, C₁₋₄ alkyl, methoxy, halomethoxy, dihalomethoxy, and trihalomethoxy.

In one preferred embodiment, R^{1a} is selected from hydrogen, hydroxy, halogen, C₁₋₈alkyl, C₂₋₈ alkenyl, C₂₋₈ alkynyl, C₃₋₆cycloalkyl, C₃₋cycloalkyl-C₁₋₃ alkyl, phenyl and C₃₋₇ heterocyclyl with optional substitution of those groups as described above. More preferably, R^{1a} is selected from hydrogen, hydroxy, halogen, C₁₋₅alkyl, phenyl and C₃₋₇ heterocyclyl with optional substitution of those groups as described above. Preferred substituents for said alkyl groups or portions of groups include groups independently selected from halogen, hydroxy, phenyl, methoxy, halomethoxy, dihalomethoxy, and trihalomethoxy. Accordingly, examples of preferred R^{1a} groups include fluoroC₁₋₁₂ alkyl, difluoroC₁₋₁₂ alkyl, and trifluoroC₁₋₁₂ alkyl. Preferred substituents for said cycloalkyl, phenyl or heterocyclyl groups or portions of groups include groups independently selected from halogen, hydroxy, C₁₋₄ alkyl, methoxy, halomethoxy, dihalomethoxy, and trihalomethoxy.

In one preferred embodiment, n is 1 and the R² group is attached to the phenyl ring at the meta-position to the position of attachment to the Y group.

R³ and R⁴ are preferably independently selected from halogen, cyano, C₁₋₄ alkyl, fluoromethyl, difluoromethyl and trifluoromethyl. More preferably, R³ and R⁴ are independently selected from halogen, methyl, fluoromethyl, difluoromethyl and trifluoromethyl. Amongst the halogens, there are preferred chlorine, bromine, and fluorine, especially chlorine and bromine, in particular bromine.

Preferably, Y is selected from oxygen or methylene. Most preferably, Y is oxygen.

W is preferably selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NH(CO)—C₀₋₃ alkylene, said alkylene, alkenylene or alkynylene groups or portions of groups optionally being substituted with 1 or 2 groups selected from hydroxy, mercapto, amino, halogen, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy.

W is more preferably selected from C₁₋₃ alkylene, C₁₋₃ alkylene-O—C₁₋₃ alkylene, C₂₋₃ alkenylene, N(R^{c})—C₁₋₂ alkylene, O—C₁₋₂ alkylene, C(O)NH—C₁₋₂ alkylene and NH(CO)—C₁₋₂ alkylene, said alkylene or alkenylene groups or portions of groups optionally being substituted with a group selected from halogen, C₁₋₂ alkyl, C₁₋₂ alkoxy, haloC₁₋₂ alkyl, dihaloC₁₋₂ alkyl, trihaloC₁₋₂ alkyl, haloC₁₋₂ alkoxy, dihaloC₁₋₂ alkoxy, and trihaloC₁₋₂ alkoxy. Amongst the halogens, there are preferred chlorine or fluorine, particularly fluorine. Most preferably, W is selected from C₁₋₃ alkylene, C₁₋₃ alkylene-O—C₁₋₃ alkylene, C(O)NH—C₁₋₂ alkylene and NH(CO)—C₁₋₂ alkylene. Most particularly preferably W is ethylene or C(O)NH—(CH₂—. Preferably the alkylene group (for example the ethylene group) is substituted with one or more halogen groups, for example one or more fluoro groups (for example one fluoro group). Monohalo C₁₋₃ alkylene (for example fluoro C₁₋₃ alkylene) thus constitutes a preferred group W.

In another preferred embodiment, W is selected from C₁₋₃ alkylene, C₂₋₃ alkenylene, C₁₋₃ alkylene-O—C₁₋₃ alkylene, O—C₁₋₃ alkylene, C(O)NH—C₁₋₂ alkylene and NH(CO)—C₁₋₂ alkylene.

R^{c} is preferably selected from hydrogen, C₁₋₂ alkyl, fluoromethyl, difluoromethyl and trifluoromethyl.

R⁵ is preferably selected from —CO₂R^{d}, —PO(OR^{d})₂, —SO₂OR^{d}, —NHSO₂R^{d}, —COCO₂R^{d} and CONR^{d}OR^{d}. More preferably, R⁵ is —CO₂R^{d}, —PO(OR^{d})₂ or —SO₂OR^{d}. Most preferably, R⁵ is —CO₂R^{d}, particularly —CO₂H.

R^{d} is preferably ethyl, methyl, hydrogen or phenyl and phenyl substituted with 1, 2 or 3 groups independently selected from amino, hydroxy, halogen and methyl. R^{d} is preferably ethyl, methyl or hydrogen, particularly hydrogen. Most preferably, R^{d} is hydrogen.

Accordingly, one preferred group of compounds of the invention includes compounds according to formula (Ia) or pharmaceutically acceptable esters, amides, solvates or salts thereof, including salts of such esters or amides, and solvates of such esters, amides or salts

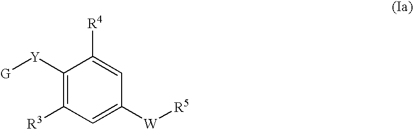

wherein:
G is a group selected from:

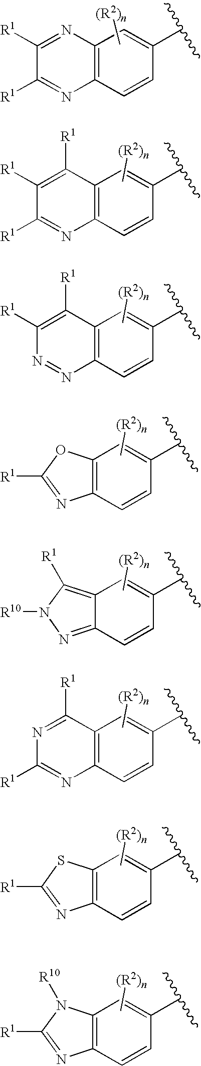

Each R¹⁰ is independently selected from —(CH₂)ₚ—S—R^{b}, —(CH₂)ₚ—SO₂—R^{b}, —(CH₂)ₚ—SO₂—NH—R^{b}, —(CH₂)ₚ—NH—CO—R^{b}, —(CH₂)ₚ—CO—NH—R^{b}, C₁₋₈alkyl, C₃₋₆ cycloalkyl, C₃₋₆cycloalkyl-C₁₋₃ alkyl, phenyl, benzyl and C₃₋₇ heterocyclyl, said alkyl groups or portions of groups optionally being substituted with 1, 2 or 3 groups each independently selected from halogen, hydroxy, N(R^{a})₂, phenyl, haloC₁₋₄alkyl, dihaloC₁₋₄alkyl, trihaloC₁₋₄alkyl, methoxy, halomethoxy, dihalomethoxy, and trihalomethoxy;
p is 1 or 2;
Each R¹⁰ is independently selected from hydrogen, hydroxy, halogen, —(CH₂)ₘ—S—R^{b}, —(CH₂)ₘ—SO₂—R^{b}, —(CH₂)ₘ—NH—SO₂—R^{b}, —(CH₂)ₘ—SO₂—NH—R^{b}, —(CH₂)ₘ—NH—CO—R^{b}, —(CH₂)ₘ—CO—NH—R^{b}, C₁₋₈ alkyl, C₃₋₆ cycloalkyl, C₃₋₆ cycloalkyl-C₁₋₃ alkyl, phenyl, benzyl and C₃₋₇ heterocyclyl, said alkyl groups or portions of groups optionally being substituted with 1, 2 or 3 groups each independently selected from halogen, hydroxy, N(R^{a})₂, phenyl, haloC₁₋₄alkyl, dihaloC₁₋₄alkyl, trihaloC₁₋₄alkyl, methoxy, halomethoxy, dihalomethoxy, and trihalomethoxy; said cycloalkyl, phenyl or heterocyclyl groups or portions of groups optionally being substituted with 1, 2 or 3 groups independently selected from halogen, hydroxy, C₁₋₄ alkyl, methoxy, halomethoxy, dihalomethoxy, and trihalomethoxy;
m is 0, 1 or 2;
R^{a} is independently selected from a hydrogen atom and a C₁₋₄ alkyl group optionally substituted with 1, 2 or 3 groups independently selected from halogen, methoxy, halomethoxy, dihalomethoxy and trihalomethoxy;
R^{b} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy and trihalomethoxy;
Each R² is independently selected from halogen, mercapto, C₁₋₄ alkoxy, C₁₋₄ alkyl and N(R^{a})₂, said alkyl or alkoxy groups or portions of groups optionally being substituted with 1, 2 or 3 groups selected from halogen, hydroxy, C₁₋₄ alkylthio, methoxy, halomethoxy, dihalomethoxy, and trihalomethoxy;
n is 0, 1 or 2;
Y is selected from oxygen, methylene, sulphur, SO, SO₂ and —N(R^{a})—;
R³ and R⁴ are independently selected from halogen, C₁₋₄ alkyl, fluoromethyl, difluoromethyl, and trifluoromethyl;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NH(CO)—C₀₋₃ alkylene, said alkylene, alkenylene or alkynylene groups or portions of groups optionally being substituted with 1 or 2 groups selected from hydroxy, mercapto, amino, halogen, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy;
R^{c} is selected from hydrogen, C₁₋₂ alkyl, fluoromethyl, difluoromethyl, and trifluoromethyl;
R⁵ is selected from —CO₂R^{d}, —PO(OR^{d})₂, —SO₂OR^{d}, —NHSO₂R^{d}, —COCO₂R^{d}, and CONR^{d}OR^{d}; and
each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₁ alkyl.

Compounds according to the invention include:
{3,5-dichloro-4-[(2-methylquinolin-6-yl)oxy]phenyl}acetic acid
3-{3,5-dibromo-4-[(2,4-dimethylquinolin-6-yl)oxy]phenyl}propanoic acid
3-{3,5-dibromo-4-[(2-methylquinolin-6-yl)oxy]phenyl}propanoic acid
3-{3,5-dibromo-4-[(4-methyl-2-propylquinolin-6-yl)oxy]phenyl}propanoic acid
3-{3,5-dibromo-4-[(2-ethylquinolin-6-yl)oxy]phenyl}propanoic acid
3-{3,5-dibromo-4-[(2-propylquinolin-6-yl)oxy]phenyl}propanoic acid
3-{3,5-dibromo-4-[(2-heptylquinolin-6-yl)oxy]phenyl}propanoic acid
3-{3,5-dibromo-4-[(4-ethyl-2-methylquinolin-6-yl)oxy]phenyl}propanoic acid
3-{3,5-dibromo-4-[(4-butyl-2-methylquinolin-6-yl)oxy]phenyl}propanoic acid
3-{3,5-dibromo-4-[(3-ethyl-2-propylquinolin-6-yl)oxy]phenyl}propanoic acid
3-{3,5-dibromo-4-[(8-chloro-4-methyl-2-propylquinolin-6-yl)oxy]phenyl}propanoic acid
{3,5-dibromo-4-[(4-methyl-2-propylquinolin-6-yl)methyl]benzyl}oxy)acetic acid
{3,5-dibromo-4-[(2,4-dimethylquinolin-6-yl)oxy]phenoxy}acetic acid
{3,5-dibromo-4-[(2-methylquinolin-6-yl)oxy]phenoxy}acetic acid
{3,5-dibromo-4-[(4-methyl-2-propylquinolin-6-yl)oxy]phenoxy}acetic acid
{3,5-dibromo-4-[(2-ethylquinolin-6-yl)oxy]phenoxy}acetic acid
(2E)-3-{3,5-dibromo-4-[(2,4-dimethylquinolin-6-yl)oxy]phenyl}acrylic acid
(2E)-3-{3,5-dibromo-4-[(4-methyl-2-propylquinolin-6-yl)oxy]phenyl}acrylic acid
3-{3,5-dichloro-4-[(2,4-dimethylquinolin-6-yl)oxy]phenyl}propanoic acid
3-{3,5-dichloro-4-[(4-methyl-2-propylquinolin-6-yl)oxy]phenyl}propanoic acid
3-{3,5-dibromo-4-[(2-methyl-1,3-benzothiazol-6-yl)oxy]phenyl}propanoic acid
{3,5-dibromo-4-[(2,4-dichloroquinolin-6-yl)oxy]phenoxy}acetic acid
{3,5-dibromo-4-[(4-chloro-2-hydroxyquinolin-6-yl)oxy]phenoxy}acetic acid
{3,5-dibromo-4-[(2,4-dihydroxyquinolin-6-yl)oxy]phenoxy}acetic acid
{3,5-dibromo-4-[(2,4-dimethoxyquinolin-6-yl)oxy]phenoxy}acetic acid
{3,5-dibromo-4-[(4-chloro-2-methoxyquinolin-6-yl)oxy]phenoxy}acetic acid
3-{3,5-dibromo-4-[(1-isopropyl-2-methyl-1H-benzimidazol-6-yl)oxy]phenyl}propanoic acid
3-{3,5-dibromo-4-[(1-ethyl-2-methyl-1H-benzimidazol-6-yl)oxy]phenyl}propanoic acid
3-{3,5-dibromo-4-[(4,8-dimethyl-2-propylquinolin-6-yl)oxy]phenyl}propanoic acid
3-{3,5-dibromo-4-[(8-hydroxy-4-methyl-2-propylquinolin-6-yl)oxy]phenyl}propanoic acid
3,5-dibromo-4-[(4-methyl-2-propylquinolin-6-yl)oxy]benzoic acid
N-{3,5-dibromo-4-[(4-methyl-2-propylquinolin-6-yl)oxy]benzoyl}glycine
3-(3,5-dibromo-4-{[4-methyl-2-propyl-8-(trifluoromethyl)quinolin-6-yl]oxy}phenyl)propanoic acid
{3,5-dibromo-4-[(2-isobutyl-1-isopropyl-1H-benzimidazol-6-yl)oxy]phenoxy}acetic acid
{3,5-dibromo-4-[(1-ethyl-2-isobutyl-1H-benzimidazol-6-yl)oxy]phenoxy}acetic acid
3-{[3,5-dibromo-4-(quinolin-6-yloxy)phenyl]amino}-3-oxopropanoic acid
3-{3,5-dibromo-4-[(4-methyl-2-propylquinolin-6-yl)oxy]phenyl}-2-fluoropropanoic acid
3-({3,5-dibromo-4-[(2-methylquinolin-6-yl)oxy]phenyl}amino)-3-oxopropanoic acid
3-({3,5-dibromo-4-[(4-methyl-2-propylquinolin-6-yl)oxy]phenyl}amino)-3-oxopropanoic acid
3-({3,5-dibromo-4-[(2-phenyl-2H-indazol-5-yl)oxy]phenyl}amino)-3-oxopropanoic acid
3-{3,5-dibromo-4-[(2-chloro-4-hydroxyquinazolin-6-yl)oxy]phenyl}propanoic acid
3-{3,5-dibromo-4-[(2-phenyl-1,3-benzothiazol-6-yl)oxy]phenyl}propanoic acid
4-(3,5-dibromo-4-{[2-(4-chlorophenyl)-1-ethyl-1H-benzimidazol-6-yl]oxy}phenyl)butanoic acid
{3,5-dichloro-4-[(2-isobutyl-1,3-benzoxazol-6-yl)oxy]phenyl}acetic acid
3-{3,5-dibromo-4-[(2-isobutyl-1-isopropyl-1H-benzimidazol-6-yl)oxy]phenyl}-2-fluoropropanoic acid
3-(3,5-dibromo-4-{[2-(cyclopentylmethyl)-1-isopropyl-1H-benzimidazol-6-yl]oxy}phenyl)-2-fluoropropanoic acid
3-{[3,5-dibromo-4-({2-[(methylamino)carbonyl]quinolin-6-yl)oxy]phenyl}amino}-3-oxopropanoic acid
3-({3,5-dibromo-4-[(2-{[(methylsulfonyl)amino]methyl}quinolin-6-yl)oxy]phenyl}amino)-3-oxopropanoic acid
3-[3,5-dibromo-4-(quinazolin-6-yloxy)phenyl]propanoic acid
3-{[3,5-dibromo-4-({2-methyl-3-[(methylamino)carbonyl]-2H-indazol-5-yl}oxy)phenyl]amino}-3-oxopropanoic acid
3-[3,5-dibromo-4-({1-ethyl-2-[2-(methylthio)ethyl]-1H-benzimidazol-6-yl}oxy)phenyl]-2-fluoropropanoic acid
3-[3,5-dibromo-4-({1-ethyl-2-[(methylsulfonyl)methyl]-1H-benzimidazol-6-yl}oxy)phenyl]-2-fluoropropanoic acid
N-{3,5-dibromo-4-[(2-phenyl-2H-indazol-5-yl)oxy]phenyl}glycine
{3,5-dibromo-4-[(2-phenyl-2H-indazol-5-yl)oxy]phenyl}acetic acid

The compounds names given above were generated in accordance with IUPAC by the ACD Labs 8.0/name program, version 8.05 and/or with ISIS DRAW Autonom 2000.

Further compounds of the invention include:
{3,5-dibromo-4-[(2-ethyl-4-methylquinolin-6-yl)methyl]phenoxy}acetic acid
(2S)-3-{3,5-dibromo-4-[(2-ethyl-4-methylquinolin-6-yl)oxy]phenyl}-2-fluoropropanoic acid
(2S)-3-[3,5-dibromo-4-({1-ethyl-2-[(methylsulfonyl)methyl]-1H-benzimidazol-6-yl}oxy)phenyl]-2-fluoropropanoic acid
{3,5-dibromo-4-[(4-methyl-2-propylquinolin-6-yl)methyl]phenoxy}acetic acid
N-{3,5-dibromo-4-[(4-methyl-2-propylquinolin-6-yl)methyl]phenyl}glycine
3-{3,5-dibromo-4-[(8-fluoro-4-methyl-2-propylquinolin-6-yl)oxy]phenyl}propanoic acid
3-{3,5-dibromor-4-[(8-fluoro-4-methyl-2-propylquinolin-6-yl)oxy]phenyl}-2-fluoropropanoic acid
{3,5-dibromo-4-[(8-fluoro-4-methyl-2-propylquinolin-6-yl)oxy]phenoxy}acetic acid
{3,5-dibromo-4-[(2-ethyl-4-methylquinolin-6-yl)methyl]phenoxy}acetic acid
3-{3,5-dibromo-4-[(2-ethyl-8-fluoro-4-methylquinolin-6-yl)oxy]phenyl}-2-fluoropropanoic acid
{3,5-dibromo-4-[(2-isobutyl-1-isopropyl-1H-benzimidazol-6-yl)methyl]phenoxy}acetic acid
N-{3,5-dibromo-4-[(1-ethyl-2-isobutyl-1H-benzimidazol-6-yl)methyl]phenyl}glycine
3-[3,5-dibromo-4-({2-[(dimethylamino)methyl]-1-ethyl-1H-benzimidazol-6-yl}oxy)phenyl]-2-fluoropropanoic acid
{3,5-dichloro-4-[(1-ethyl-2-isobutyl-1H-benzimidazol-6-yl)oxy]phenyl}acetic acid
3-{3,5-dibromo-4-[(2-ethyl-4-methylquinazolin-6-yl)oxy]phenyl}-2-fluoropropanoic acid
3-{3,5-dibromo-4-[(3,4-dimethylcinnolin-6-yl)oxy]phenyl}-2-fluoropropanoic acid
3-{3,5-dibromo-4-[(2-isobutyl-7-methyl-1,3-benzothiazol-6-yl)oxy]phenyl}-2-fluoropropanoic acid
{3,5-dichloro-4-[(2-isobutyl-7-methyl-1,3-benzothiazol-6-yl)oxy]phenyl}acetic acid
3-{3,5-dibromo-4-[(7-fluoro-2-isobutyl-1,3-benzoxazol-6-yl)oxy]phenyl}-2-fluoropropanoic acid
[3,5-dibromo-4-({1-ethyl-2-[(methylsulfonyl)methyl]-1H-benzimidazol-6-yl}methyl)phenoxy]acetic acid
[3,5-dichloro-4-({1-ethyl-2-[(methylsulfonyl)methyl]-1H-benzimidazol-6-yl}oxy)phenyl]acetic acid
(4-{[2-(acetylamino) 1-ethyl-1H-benzimidazol-6-yl]methyl}-3,5-dibromophenoxy)acetic acid
(4-{[2-(acetylamino) 1-ethyl-1H-benzimidazol-6-yl]oxy}-3,5-dibromophenoxy)acetic acid
3-(4-{[2-(acetylamino)-1-ethyl-1H-benzimidazol-6-yl]oxy}-3,5-dibromophenyl)-2-fluoropropanoic acid
3-[3,5-dibromo-4-({1-ethyl-2-[(methylsulfonyl)amino]-1H-benzimidazol-6-yl}oxy)phenyl]-2-fluoropropanoic acid
3-[3,5-dibromo-4-({1-ethyl-2-[(ethylamino)carbonyl]-1H-benzimidazol-6-yl}oxy)phenyl]-2-fluoropropanoic acid
3-[3,5-dibromo-4-({1-ethyl-2-[2-(methylamino)-2-oxoethyl]-1H-benzimidazol-6-yl}oxy)phenyl]-2-fluoropropanoic acid
3-[3,5-dibromo-4-({2-[2-(dimethylamino)-2-oxoethyl]-1-ethyl-1H-benzimidazol-6-yl}oxy)phenyl]-2-fluoropropanoic acid
[3,5-dibromo-4-({1-ethyl-2-[2-(methylamino)-2-oxoethyl]-1H-benzimidazol-6-yl}oxy)phenoxy]acetic acid
{3,5-dibromo-4-[(3-ethyl-2-isobutyl-2H-indazol-5-yl)methyl]phenoxy}acetic acid
3-{3,5-dibromo-4-[(3-ethyl-2-isobutyl-2H-indazol-5-yl)oxy]phenyl}-2-fluoropropanoic acid
3-{3,5-dibromo-4-[(2-isobutyl-3-methyl-2H-indazol-5-yl)oxy]phenyl}-2-fluoropropanoic acid
N-{3,5-dibromo-4-[(3-methyl-2-phenyl-2H-indazol-5-yl)oxy]phenyl}glycine
N-{3,5-dibromo-4-[(3-ethyl-2-isobutyl-2H-indazol-5-yl)oxy]phenyl}glycine
{3,5-dichloro-4-[(3-ethyl-2-isobutyl-2H-indazol-5-yl)oxy]phenyl}acetic acid
N-{3,5-dibromo-4-[(2-isobutyl-3-methyl-2H-indazol-5-yl)oxy]phenyl}glycine
N-[3,5-dibromo-4-({3-methyl-2-[2-(methylamino)-2-oxoethyl]-2H-indazol-5-yl}oxy)phenyl]glycine
N-[3,5-dibromo-4-({3-methyl-2-[(methylsulfonyl)methyl]-2H-indazol-5-yl}oxy)phenyl]glycine
N-{3,5-dibromo-4-[(2-ethyl-8-fluoro-4-methylquinolin-6-yl)oxy]benzoyl}glycine
3-({3,5-dibromo-4-[(2-ethyl-4-methylquinolin-6-yl)methyl]phenyl}amino)-3-oxopropanoic acid
3-({3,5-dibromo-4-[(2-isobutyl-1-methyl-1H-benzimidazol-6-yl)methyl]phenyl}amino)-3-oxopropanoic acid
N-{3,5-dibromo-4-[(1-ethyl-2-isobutyl-1H-benzimidazol-6-yl)oxy]benzoyl}glycine
N-{3,5-dibromo-4-[(2-ethyl-4-methylquinazolin-6-yl)oxy]benzoyl}glycine
N-{3,5-dibromo-4-[(1-ethyl-2-isobutyl-1H-benzimidazol-6-yl)oxy]benzoyl}alanine
N-{3,5-dibromo-4-[(2-ethyl-4-methylquinazolin-6-yl)oxy]benzoyl}alanine
N-{3,5-dibromo-4-[(3,4-dimethylcinnolin-6-yl)oxy]benzoyl}glycine
3-({3,5-dibromo-4-[(2-isobutyl-7-methyl-1,3-benzothiazol-6-yl)oxy]phenyl}amino)-3-oxopropanoic acid
N-{3,5-dibromo-4-[(3,4-dimethylcinnolin-6-yl)oxy]benzoyl}valine
N-{3,5-dibromo-4-[(7-fluoro-2-isobutyl-1,3-benzoxazol-6-yl)oxy]benzoyl}alanine
3-[(4-{[2-(acetylamino-1-ethyl-1H-benzimidazol-6-yl]methyl}-3,5-dibromophenyl)amino]-3-oxopropanoic acid
N-(4-{[2-(acetylamino)-1-ethyl-1H-benzimidazol-6-yl]oxy}-3,5-dibromobenzoyl)glycine
N-[3,5-dibromo-4-({1-ethyl-2-[(methylamino)carbonyl]-1H-benzimidazol-6-yl}oxy)benzoyl]glycine
3-{[3,5-dibromo-4-({2-[2-(dimethylamino)-2-oxoethyl]-1-ethyl-1H-benzimidazol-6-yl}oxy)phenyl]amino}-3-oxopropanoic acid
3-({3,5-dibromo-4-[(3-ethyl-2-isobutyl-2H-indazol-5-yl)methyl]phenyl}amino)-3-oxopropanoic acid
N-{3,5-dibromo-4-[(3-ethyl-2-isobutyl-2H-indazol-5-yl)oxy]benzoyl}glycine
N-{3,5-dibromo-4-[(3-ethyl-2-isopropyl-2H-indazol-5-yl)oxy]benzoyl}alanine
3-{[3,5-dibromo-4-({3-methyl-2-[(methylsulfonyl)methyl]-2H-indazol-5-yl}oxy)phenyl]amino}-3-oxopropanoic acid
N-[3,5-dibromo-4-({3-methyl-2-[(methylsulfonyl)methyl)-2H-indazol-5-yl}oxy)benzoyl]glycine
3-{3,5-bromo-4-(1-ethyl-2-isobutyl-1H-benzimidazol-6-yl)oxy]phenyl}-2,2-difluoropropanoic acid
{4-[(1-ethyl-2-isobutyl-1H-benzimidazol-6-yl)oxy]-3,5-bis(trifluoromethyl)phenyl}acetic acid
2-chloro-3-{3,5dibromo-4-[(1-ethyl-2-isobutyl-1H-benzimidazol-6-yl)oxy]phenyl}propanoic acid
   Most favoured compounds are:
{3,5-bromo-4-[(2-ethyl methylquinolin-6-yl)methyl]phenoxy}acetic acid
(2S)-3-{3,5-bromo-4-[(2-ethyl-4-methylquinolin-6-yl)oxy]phenyl}-2-fluoropropanoic acid
(2S)-3-[3,5-dibromo-4-({1-ethyl-2-[(methylsulfonyl)methyl]-1H-benzimidazol-6-yl}oxy)phenyl]-2-fluoropropanoic acid
{3,5-dibromo-4-[(4-methyl-2-propylquinolin-6-yl)methyl]phenoxy}acetic acid
N-{3,5-dibromo-4-[(4-methyl-2-propylquinolin)methyl]phenyl}glycine
3-{3,5-dibromo-4-[(8-fluoro-4-methyl-2-propylquinolin-6-yl)oxy]phenyl}propanoic acid
3-{3,5-dibromo-4-[(8-fluoro-4-methyl-2-propylquinolin-6-yl)oxy]phenyl}-2-fluoropropanoic acid
{3,5-bromo-4-[(8-fluoro-4-methyl-2-propylquinolin-6-yl)oxy]phenoxy}acetic acid
{3,5dibromo-4-[(2-ethyl-4-methylquinolin-6-yl)methyl]phenoxy}acetic acid
3-{3,5-dibromo-4-[(2-ethyl-8-fluoro-4-methylquinolin-6-yl)oxy]phenyl}-2-fluoropropanoic acid
{3,5-dibromo-4-[(2-isobutyl-1-isopropyl-1H-benzimidazol-6-yl)methyl]phenoxy}acetic acid
N-{3,5dibromo-4-[(1-ethyl-2-isobutyl-1H-benzimidazol-6-yl)methyl]phenyl}glycine
3-[3,5-dibromo-4-({2-[(dimethylamino)methyl]-1-ethyl-1H-benzimidazol-6-yl}oxy)phenyl]-2-fluoropropanoic acid
{3,5-dichloro-4-[(1-ethyl-2-isobutyl-1H-benzimidazol-6-yl)oxy]phenyl}acetic acid
3-{3,5-dibromo-4-[(2-ethyl-4-methylquinazolin-6-yl)oxy]phenyl}-2-fluoropropanoic acid
3-{3,5-dibromo-4-[(3,4-dimethylcinnolin-6-yl)oxy]phenyl}-2-fluoropropanoic acid
3-{3,5-dibromo-4-[(2-isobutyl-7-methyl-1,3-benzothiazol-6-yl)oxy]phenyl}-2-fluoropropanoic acid
{3,5-dichloro-4-[(2-isobutyl-7-methyl-1,3-benzothiazol-6-yl)oxy]phenyl}acetic acid
3-{3,5dibromo-4-[(7-fluoro-2-isobutyl-1,3-benzoxazol-6-yl)oxy]phenyl}-2-fluoropropanoic acid
[3,5-dibromo-4-({1-ethyl-2-[(methylsulfonyl)methyl]-1H-benzimidazol-6-yl}methyl)phenoxy]acetic acid
[3,5-dichloro-4-({1-ethyl-2-[(methylsulfonyl)methyl]-1H-benzimidazol-6-yl}oxy)phenyl]acetic acid
(4-{[2-(acetylamino)-1-ethyl-1H-benzimidazol-6-yl]methyl}-3,5dibromophenoxy)acetic acid
(4-{[2-(acetylamino)-1-ethyl-1H-benzimidazol-6-yl]oxy}-3,5-dibromophenoxy)acetic acid
3-(4-{[2-acetylamino)-1-ethyl-1H-benzimidazol-6-yl]oxy}-3,5-bromophenyl)-2-fluoropropanoic acid
3-[3,5-dibromo-4-({1-ethyl-2-[(methylsulfonyl)amino]-1H-benzimidazol-6-yl}oxy)phenyl]-2-fluoropropanoic acid
3-[3,5-dibromo-4-({1-ethyl-2-[(ethylamino)carbonyl]-1H-benzimidazol-6-yl}oxy)phenyl]-2-fluoropropanoic acid
3-[3,5-dibromo-4-({-ethyl-2-[2-(methylamino)-2-oxoethyl]-1H-benzimidazol-6-yl}oxy)phenyl]-2-fluoropropanoic acid
3-[3,5-dibromo-4-({2-[2-(dimethylamino)-2-oxoethyl]-1-ethyl-1H-benzimidazol-6-yl}oxy)phenyl]-2-fluoropropanoic acid
[3,5-dibromo-4-({1-ethyl-2-[2-(methylamino)-2-oxoethyl]-1H-benzimidazol-6-yl}oxy)phenoxy]acetic acid
{3,5-dibromo-4-[(3-ethyl-2-isobutyl-2H-indazol-5-yl)methyl]phenoxy}acetic acid
3-{3,5-dibromo-4-[(3-ethyl-2-isobutyl-2H-indazol-5-yl)oxy]phenyl}-2-fluoropropanoic acid
3-{3,5-dibromo-4-[(2-isobutyl-3-methyl-2H-indazol-5-yl)oxy]phenyl}-2-fluoropropanoic acid
N-{3,5-dibromo-4-[(3-methyl-2-phenyl-2H-indazol-5-yl)oxy]phenyl}glycine
N-{3,5-dibromo-4-[(3-ethyl-2-isobutyl-2H-indazol-5-yl)oxy]phenyl}glycine
{3,5-dichloro-4-[(3-ethyl-2-isobutyl-2H-indazol-5-yl)oxy]phenyl}acetic acid
N-{3,5-dibromo-4-[(2-isobutyl-3-methyl-2H-indazol-5-yl)oxy]phenyl}glycine
N-[3,5-dibromo-4-({3-methyl-2-[2-(methylamino)-2-oxoethyl]-2H-indazol-5-yl}oxy)phenyl]glycine
N-[3,5-dibromo-4-({3-methyl-2-[(methylsulfonyl)methyl]-2H-indazol-5-yl}oxy)phenyl]glycine

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. According to the present invention, examples of physiologically functional derivatives include esters, amides, and carbamates; preferably esters and amides.

Suitable salts according to the invention include those formed with organic or inorganic acids or bases. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases, for example dicyclohexylamine and N-methyl-D-glucomine.

Pharmaceutically acceptable esters and amides of the compounds of formula (I) may have an appropriate group, for example an acid group, converted to a C₁₋₄ alkyl, C₅₋₁₀ aryl, C₅₋₁₀ aryl-C₁₋₆alkyl, or amino acid ester or amide. Pharmaceutically acceptable esters of the compounds of formula (I) may have an appropriate group, for example a hydroxy group, converted to a C₁₋₄ alkyl, C₅₋₁₀ aryl, or C₅₋₁₀ aryl-C₁₋₄ alkyl ester. Pharmaceutically acceptable amides and carbamates of the compounds of formula (I) may have an appropriate group, for example an amino group, converted to a C₁₋₄ alkyl, C₅₋₁₀ aryl, C₅₋₁₀ aryl-C₁₋₆ alkyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); and in Edward B. Roche, ed., Bioreversible Carriers in Drug Design, American Pharmaceutical Association and Pergamon Press, 1987, both of which are incorporated herein by reference.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl, and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethylbutyl, and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkynyl groups include ethenyl, 1-propenyl and 2-propenyl.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo[2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred. In some embodiments, fluorine is especially preferred. In alternative embodiments, chlorine or bromine are especially preferred.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole. Examples of preferred heterocyclyl groups include piperidinyl, tetrahydrofuranyl, tetrahydropyranyl, pyridyl, pyrimidyl and indolyl.

As used herein the term “cycloalkylalkyl” means a group cycloalkyl-alkyl-attached through the alkyl group, “cycloalkyl” and “alkyl” being understood to have the meanings outlined above.

As mentioned above, the compounds of the invention have activity as thyroid receptor ligands. The compounds of the invention are preferably selective agonists or partial agonists of the thyroid receptor. Preferably compounds of the present invention possess activity as agonists of the thyroid receptor, preferably selective agonists of the thyroid receptor-beta. They may thus be used in the treatment of diseases or disorders associated with thyroid receptor activity, particularly diseases or disorders for which selective agonists of the thyroid receptor-beta are indicated. In particular, compounds of the present invention may be used in the treatment of diseases or disorders associated with metabolism dysfunction or which are dependent upon the expression of a T₃ regulated gene.

Clinical conditions for which an agonist or partial agonist is indicated include, but are not limited to, hypothyroidism; subclinical hyperthyroidism; non-toxic goiter; atherosclerosis; thyroid hormone replacement therapy (e.g., in the elderly); malignant tumor cells containing the thyroid receptor; papillary or follicular cancer; maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); accelerating healing of complicated fractures (e.g. distraction osteogenesis); in joint replacement; eating disorders (e.g., anorexia); treatment of obesity and growth retardation associated with obesity; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of hyperinsulinemia; stimulation of osteoblasts, bone remodeling and cartilage growth; regulation of food intake; treatment of insulin resistance, including NIDDM, in mammals (e.g., humans); treatment of insulin resistance in the heart; treatment of congestive heart failure; treatment of musculoskeletal impairment (e.g., in the elderly); improvement of the overall pulmonary function; skin disorders or diseases, such as dermal atrophy, glucocorticoid induced dermal atrophy, including restoration of dermal atrophy induced by topical glucocorticoids, and the prevention of dermal atrophy induced by topical glucocorticoids (such as the simultaneous treatment with topical glucocorticoid or a pharmacological product including both glucocorticoid and a compound of the invention), the restoration/prevention of dermal atrophy induced by systemic treatment with glucocorticoids, restoration/prevention of atrophy in the respiratory system induced by local treatment with glucocorticoids, UV-induced dermal atrophy, dermal atrophy induced by aging (wrinkles, etc.), wound healing, post surgical bruising caused by laser resurfacing, keloids, stria, cellulite, roughened skin, actinic skin damage, lichen planus, ichtyosis, acne, psoriasis, Demier's disease, eczema, atopic dermatitis, chloracne, pityriasis and skin scarring. In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention. The term treatment includes, where appropriate, prophylactic treatment.

The compounds of the invention find particular application in the treatment or prophylaxis of the following: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

The compounds of the invention find especial application in the treatment or prophylaxis of the following: (1) hypercholesterolemia, dyslipidemia or any other lipid disorder manifested by an unbalance of blood or tissue lipid levels; (2) atherosclerosis; (3) obesity; (4) diabetes.

The invention also provides a method for the treatment or prophylaxis of a condition in a mammal mediated by a thyroid receptor, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt. Clinical conditions mediated by a thyroid receptor that may be treated by the method of the invention are those described above.

The invention also provides the use of a compound of formula (I) as defined above or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by a thyroid receptor. Clinical conditions mediated by a thyroid receptor that may be treated by the method of the invention are those described above.

Hereinafter, the term “active ingredient” means a compound of formula (I) as defined above, or a pharmaceutically acceptable ester, amide, solvate or salt thereof, including a salt of such an ester or amide, and a solvate of such an ester, amide or salt.

The amount of active ingredient which is required to achieve a therapeutic effect will, of course, vary with the particular compound, the route of administration, the subject under treatment, and the particular disorder or disease being treated. The compounds of the invention may be administered orally or via injection at a dose of from 0.1 to 1500 mg/kg per day, preferably 0.1 to 500 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation or composition. Accordingly, the invention provides a pharmaceutical formulation comprising a compound of formula (I) as defined above or a pharmaceutically acceptable ester, amide, solvate or salt thereof, including a salt of such an ester or amide, and a solvate of such an ester, amide or salt, and a pharmaceutically acceptable excipient. Pharmaceutical compositions of the invention may take the form of a pharmaceutical formulation as described below.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divide solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of formula (I) can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an anti-dyslipidemic agent or other pharmaceutically active material.

The compounds of the present invention may be employed in combination with one or more other modulators and/or ligands of the thyroid receptor or one or more other suitable therapeutic agents selected from the group consisting of cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

Examples of suitable hypolipidemic agents for use in combination with the compounds of the present invention include an acyl coenzyme A cholesterol acyltransferase (ACAT) inhibitor, a microsomal triglyceride transfer protein (MTP) inhibitor, a cholesterol ester transfer protein (CETP) inhibitor, a ileal bile acid transporter (IBAT) inhibitor, any cholesterol absorption inhibitor, a 3-hydroxy-3-methylglutaryl coenzyme A (HMG-CoA) reductase inhibitor, a squalene synthetase inhibitor, a bile acid sequestrant, a peroxisome proliferator-activator receptor (PPAR)-alpha agonist, a peroxisome proliferator-activator receptor (PPAR)-delta agonist, any peroxisome proliferator-activator receptor (PPAR)-gamma/delta dual agonist, any peroxisome proliferator-activator receptor (PPAR)-alpha/delta dual agonist, a nicotinic acid or a derivative thereof, and a thiazolidinedione or a derivative thereof.

Examples of suitable hypolipidemic agents for use in combination with the compounds of the present invention also include ezetimibe, simvastatin, atorvastatin, rosuvastatin, cerivastatin, fluvastatin, lovastatin, pravastatin, fenofibrate, gemfibrozil and bezafibrate.

Examples of suitable anti-diabetic agents for use in combination with the compounds of the present invention include biguanides (e.g., metformin or phenformin), glucosidase inhibitors (e.g., acarbose or miglitol), insulins (including insulin secretagogues or insulin sensitizers), meglitinides (e.g., repaglinide), sulfonylureas (e.g., glimepiride, glyburide, glipyride, gliclazide, chlorpropamide and glipizide), biguanide/glyburide combinations (e.g., Glucovance®), thiazolidinediones (e.g., troglitazone, rosiglitazone, englitazone, darglitazone and pioglitazone), PPAR-alpha agonists, PPAR-gamma agonists, PPAR alpha/gamma dual agonists, PPAR alpha/delta dual agonists, SGLT 1, 2 or 3 inhibitors, glycogen phosphorylase inhibitors, inhibitors of fatty acid binding protein (aP2), glucagon-like peptide-1 (GLP-1), glucocorticoid (GR) antagonist and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L₇₅₀₃₅₅ (Merck), or CP331648 (Pfizer) or other known beta δ agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and GB98/284425 (KaroBio), CB-1 (cannabinoid receptor) antagonists (see G. Colombo et al, “Appetite Suppression and Weight Loss After the Cannabinoid Antagonist SR 141716”, Life Sciences, Vol 63, PL 113-117 (1998)) and/or an anorectic agent, such as dexamphetamine, phentermine, phenylpropanolamine or mazindol.

The compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-I and IGF-2, or with alpha-adrenergic agonists, such as clonidine or serotinin 5-HT_{D} agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®), integrin antagonists, alpha-4 beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1).

Examples of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cardiac glycosides for use in combination with the compounds of the present invention include digitalis and ouabain.

Examples of suitable cholesterol/lipid lowering agents for use in combination with the compounds of the present invention include HMG-CoA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

MTP inhibitors which may be employed herein in combination with one or more compounds of formula (I) include MTP inhibitors as disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 all incorporated herein by reference.

The HMG CoA reductase inhibitors which may be employed in combination with one or more compounds of formula (I) include mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Further HMG CoA reductase inhibitors which may be employed herein include fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No. 0,142,146 A2, as well as other known HMG CoA reductase inhibitors.

The squalene synthetase inhibitors which may be used in combination with the compounds of the present invention include, but are not limited to, O-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinylmethyl)phosphonates, terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, as well as other squalene synthetase inhibitors as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996).

Bile acid sequestrants which may be used in combination with the compounds of the present invention include cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, paminosalicylic acid, aspirin, poly(diallylmethylamine) derivatives such as disclosed in U.S. Pat. No. 4,759,923, quaternary amine poly(diallyidimethylammonium chloride) and ionenes such as disclosed in U.S. Pat. No. 4,027,009, and other known serum cholesterol lowering agents.

ACAT inhibitors suitable for use in combination with compounds of the invention include ACAT inhibitors as described in, Drugs of the Future 24, 9-15 (1999), (Avasimibe); “The ACAT inhibitor, C1-0111 is effective in the prevention and regression of aortic fatty streak area in hamsters”, Nicolosi et at, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62.

Examples of suitable cholesterol absorption inhibitor for use in combination with the compounds of the invention include SCH₄₈₄₆₁ (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostaglandins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

For the treatment of skin disorders or diseases as described above, the compounds of the present invention may be used alone or optionally in combination with a retinoid, such as tretinoin, or a vitamin D analog.

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Lett., 9, 1003-1008 (1999) and Hamann, L. G. et al., J. Med. Chem., 42, 210-212 (1999).

A further use of the compounds of this invention is in combination with steroidal or non-steroidal progesterone receptor agonists (“PRA”), such as levonorgestrel, medroxyprogesterone acetate (MPA).

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with a hypolipidemic agent, an antidepressant, a bone resorption inhibitor and/or an appetite suppressant, the compounds of formula (I) may be employed in a weight ratio to the additional agent within the range from about 500:1 to about 0.005:1, preferably from about 300:1 to about 0.01:1.

Where the antidiabetic agent is a biguanide, the compounds of formula (I) may be employed in a weight ratio to biguanide within the range from about 0.01:1 to about 100:1, preferably from about 0.5:1 to about 2:1.

The compounds of formula (I) may be employed in a weight ratio to a glucosidase inhibitor within the range from about 0.01:1 to about 100:1, preferably from about 0.5:1 to about 50:1.

The compounds of formula (I) may be employed in a weight ratio to a sulfonylurea in the range from about 0.01:1 to about 100:1, preferably from about 0.2:1 to about 10:1.

The compounds of formula (I) may be employed in a weight ratio to a thiazolidinedione in an amount within the range from about 0.01:1 to about 100:1, preferably from about 0.5:1 to about 5:1. The thiazolidinedione may be employed in amounts within the range from about 0.01 to about 2000 mg/day, which may optionally be administered in single or divided doses of one to four times per day. Further, where the sulfonylurea and thiazolidinedione are to be administered orally in an amount of less than about 150 mg, these additional agents may be incorporated into a combined single tablet with a therapeutically effective amount of the compounds of formula (I).

Metformin, or salt thereof, may be employed with the compounds of formula (I) in amounts within the range from about 500 to about 2000 mg per day, which may be administered in single or divided doses one to four times daily.

The compounds of formula (I) may be employed in a weight ratio to a PPAR-alpha agonist, a PPAR-gamma agonist, a PPAR-alpha/gamma dual agonist, an SGLT2 inhibitor and/or an aP2 inhibitor within the range from about 0.01:1 to about 100:1, preferably from about 0.5:1 to about 5:1.

An MTP inhibitor may be administered orally with the compounds of formula (I) in an amount within the range of from about 0.01 mg/kg to about 100 mg/kg and preferably from about 0.1 mg/kg to about 75 mg/kg, one to four times daily. A preferred oral dosage form, such as tablets or capsules, may contain the MTP inhibitor in an amount of from about 1 to about 500 mg, preferably from about 2 to about 400 mg, and more preferably from about 5 to about 250 mg, administered on a regimen of one to four times daily. For parenteral administration, the MTP inhibitor may be employed in an amount within the range of from about 0.005 mg/kg to about 10 mg/kg and preferably from about 0.005 mg/kg to about 8 mg/kg, administered on a regimen of one to four times daily.

A HMG CoA reductase inhibitor may be administered orally with the compounds of formula (I) within the range of from about 1 to 2000 mg, and preferably from about 4 to about 200 mg. A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

A squalene synthetase inhibitor may be administered with the compounds of formula (I) within the range of from about 10 mg to about 2000 mg and preferably from about 25 mg to about 200 mg. A preferred oral dosage form, such as tablets or capsules, will contain the squalene synthetase inhibitor in an amount of from about 10 to about 500 mg, preferably from about 25 to about 200 mg.

The compounds of formula (I) as described above also find use, optionally in labelled form, as a diagnostic agent for the diagnosis of conditions associated with malfunction of the thyroid receptor. For example, such a compound may be radioactively labelled.

The compounds of formula (I) as described above, optionally in labelled form, also find use as a reference compound in methods of discovering other antagonists or partial antagonists of the thyroid receptor. Thus, the invention provides a method of discovering a ligand of the thyroid receptor which comprises use of a compound of the invention or a compound of the invention in labelled form, as a reference compound. For example, such a method may involve a competitive binding experiment in which binding of a compound of formula (I) to the thyroid receptor is reduced by the presence of a further compound which has thyroid receptor-binding characteristics, for example stronger thyroid receptor-binding characteristics than the compound of formula (I) in question.

Numerous synthetic routes to the compounds of the present invention can be devised by any person skilled in the art and the possible synthetic routes described below do not limit the invention. Many methods exist in the literature for the synthesis of diaryl ethers, for example, two references directly apply to the synthesis of thyroid hormone analogs: Evans D. A. et al. Tetrahedron Lett., 39, 2937-2940, 1998 and Salamonczyk G. M. et al., Tetrahedron Lett., 38, 6965-6968, 1997.

In particular, methods for synthesizing compounds of formula (I) in which Y is —N(R^{a})—, sulphur, and methylene are generally described in the literature (Y is —N(R^{a})—: Chan D. M. T. et al., Tetrahedron Lett., 39, 2933-2936, 1998; Wolfe J. P. et al., J. Am. Chem. Soc., 118, 7215, 1996; Driver, M. S., Hartwig, J. F., J. Am. Chem. Soc., 118, 7217, 1996; see references in the review by Frost, C. G., Mendonce, P., J. Chem. Soc. Perkin. 1, 2615-2623, 1998; Y is S: Harrington, C. R., Biochem. J., 43, 434-437, 1948; Dibbo, A. et al., J. Chem. Soc., 2890-2902, 1961; Yokoyama, N. et al., U.S. Pat. No. 5,401,772, 1995; Y is CH₂: Horner, L., Medem, H. H. G., Chem. Ber., 85, 520-530, 1952; Chiellini, G. et al., Chemistry & Biology, 5, 299-306, 1998). Compounds wherein Y is SO or SO₂ may be synthesised from the corresponding compound in which Y is sulphur by oxidation with a suitable oxidising agent.

The invention also provides a method for preparing a compound of formula (I) in accordance with the invention as described above wherein Y is selected from oxygen, sulphur, SO, SO₂ and —N(R^{a})—, comprising a step of reacting
a compound of formula (II)

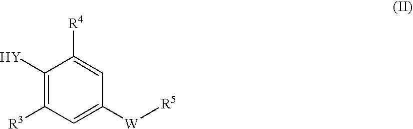

wherein W, R³, R⁴, and R⁵ are as defined above and Y is selected from oxygen, sulphur, and —N(R^{a})—
with a compound of formula (III)

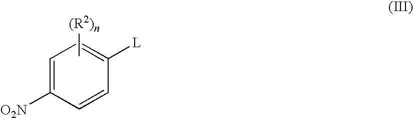

wherein R² is as defined above and L is a suitable leaving group, optionally in the presence of a suitable base and, optionally, in the presence of copper powder, followed by reduction of the nitro group to an amino group using a suitable reducing agent, followed by interconversion to a compound of formula (I).

Suitable leaving groups L include triflate, mesilate and halogen, for example a fluoride. Suitable bases include carbonates, for example potassium carbonate or cesium carbonate, alkylamines, for example diisopropylamine or triethylamine, and alkali metal hydroxides for example potassium hydroxide or sodium hydroxide. Other combinations of leaving groups and bases may be employed, as is known by the person skilled in the art. Optionally, one or more coupling reagents may be employed. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Suitable reducing agents include hydrogen and a platinum oxide catalyst, iron in a suitable acid, for example hydrochloric acid, or SnCl₂ in ethanol. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (II) include:
3-(3,5-Dibromo-4-hydroxy-phenyl)-propionic acid methyl ester
(E)-3-(3,5-Dibromo-4-hydroxy-phenyl)-acrylic acid methyl ester
(3,5-Dibromo-4-hydroxy-phenoxy)acetic acid methyl ester
3-(3,5-Dibromo-4-hydroxy-phenyl)-2-fluoro-propionic acid methyl ester
(3,5-Dibromo-4-hydroxy-benzoylaminoyacetic acid methyl ester
N-(3,5-Dibromo-4-hydroxy-phenyl)-malonamic acid methyl ester
   Preferred compounds of formula (III) include:
1-Fluoro-4-nitro-benzene
2-Chloro-4-fluoro-1-nitro-benzene

The invention also provides a method for preparing a compound of formula (I) in accordance with the invention as described above wherein G is the following group:

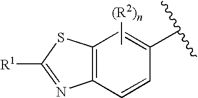

comprising a step of reacting
a compound of formula (IV)

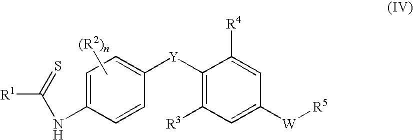

wherein W, Y, R¹, R², R³, R⁴ and R⁵ are as defined above
with a suitable oxidising agent in the presence of a suitable base, followed optionally by interconversion to another compound as defined in formula (I).

Suitable oxidising agents for use in the reaction include potassium hexacyanoferrate(III) K₃Fe(CN)₆.

Suitable bases for use in the reaction include metal hydroxides, for example sodium hydroxide, lithium hydroxide, or potassium hydroxide. Other bases may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Other suitable conditions and reagents suitable for use in the above reactions for the preparation of compounds of formula (I) in accordance with the invention or for the synthesis of intermediates suitable for preparing compounds of formula (I) are described in the following references: Yarovenko V. N., Stoyanovich F. M., Zolotarskaya O. Yu., Chernoburova E. I., Zavarzin I. V. and Krayushkin M. M., Russian Chemical Bulletin, International Edition, 51, No. 1, 2002, 144-147. Misra T., Ganguly T., Kamila S., Basu C., De A. Spectrochimica Acta, Part A 57, 2001, 2795-2808
Gallagher T., Pardoe D. A. and Porter R. A., Tetrahedron Lett., 41, 2000, 5415-5418.
Martin-Smith, M., Gates, M., J. Am. Chem. Soc., 78, 1956, 5351-5357.
Martin-Smith, M., Gates, M., J. Am. Chem. Soc., 78, 1956, 6177-6180.
   Preferred compounds of formula (IV) include:
3-[3,5-Dibromo-4-(4-thioacetylamino-phenoxy)-phenyl]-propionic acid methyl ester
   The invention also provides a method for preparing a compound of formula (I) in accordance with the invention as described above wherein G is the following group:

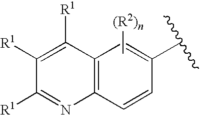

comprising a step of reacting
a compound of formula (V)

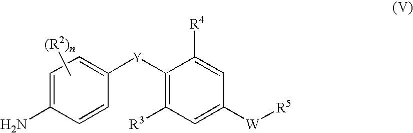

wherein R², R³, R⁴, R⁵, Y and W are as defined above
with a a compound of formula (VI)

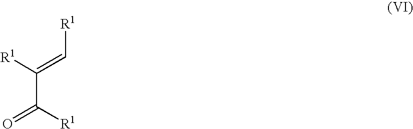

wherein R¹ is as defined above
in the presence of a suitable acid, followed optionally by interconversion to another compound of formula (I).

Suitable acids for use in the reaction include hydrochloric acid.

Other acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Other suitable conditions and reagents suitable for use in the above reactions for the preparation of compounds of formula (I) in accordance with the invention or for the synthesis of intermediates suitable for preparing compounds of formula (I) are described in the following references:
P. Riehm et al., Ber., 1885, 18, 2245; 1886, 19, 1394; idem, Ann., 1887, 238, 9.
R. G. Gould, W. A. Jacobs, J. Am. Chem. Soc., 1939, 61, 2890.
L. Knorr, Ann., 1886, 236, 69; 1888, 245, 357, 378.
J. Polanski, F. Zouhiri, L. Jeanson, D. Desmaele, J. d'Angelo, J.-F. Mouscadet, R. Gieleciak, J. Gasteiger and M. Le Bret, J. Med. Chem., 2002, 45, 4647.
H. Z. Syeda Huma, R. Halder, S. Singh Kalra, J. Das and J. Iqbala, Tetrahedron Lett., 2002, 43, 6485.
J. S. Yadav, B. V. S. Reddy, R. Srinivasa Rao, V. Naveenkumar, K. Nagaiah Synthesis, 2003, 10, 1610.
A. G. Osborne, J. M. Buley, H. Clarke, R. C. H. Dakin and P. I. Priceb, J. Chem. Soc. Perkin Trans. 1; 1993, 2747.
Mabire, D.; Coupa, S.; Adelinet, C.; Poncelet, A.; Simonnet, Y.; Venet, M.; Wouters, R.; Lesage, A. S. J.; Beijsterveldt, L. V.; Bischoff, F.; J. Med. Chem.; 2005; 48(6), 2134.
   Preferred compounds of formula (V) include:
3-[4-(4-Amino-phenoxy)-3,5-dibromo-phenyl]-propionic acid methyl ester
3-[4-(4-Amino-phenoxy)-3,5-dibromo-phenyl]-2-fluoro-propionic acid methyl ester
(E)-3-[4-(4-Amino-phenoxy)-3,5-dibromo-phenyl]-acrylic acid methyl ester
[4-(4-Amino-phenoxy)-3,5-dibromo-phenoxy]-acetic acid methyl ester
5 [4-(4-Amino-phenoxy)-3,5-dibromo-benzoylamino]-acetic acid methyl ester
N-[4-(4-Amino-phenoxy)-3,5-dibromo-phenyl]-malonamic acid methyl ester
   Preferred compounds of formula (VI) include:
(E)-Hept-3-en-2-one
(E)-Hex-3-en-2-one
(E)-Hept-4-en-3-one
   The invention also provides a method for preparing a compound of formula (I) in accordance with the invention as described above wherein G is the following group:

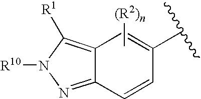

comprising a step of reacting
a compound of formula (VII)

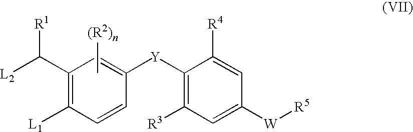

wherein R¹, R², R³, R⁴, R⁵, Y and W are as defined above and L₁ and L₂ are suitable leaving groups;
with a hydrazine compound of formula (VIII)

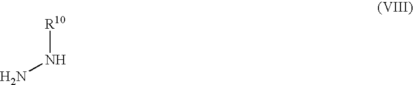

wherein R¹⁰ is as defined above, followed optionally by interconversion to another compound of formula (I).

Suitable leaving groups L₁ and L₂ include halogens, for example a bromide.

Other leaving groups may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Other suitable conditions and reagents suitable for use in the above reactions for the preparation of compounds of formula (I) in accordance with the invention or for the synthesis of intermediates suitable for preparing compounds of formula (I) are described in the following references:
L. G. Fedenok and N. A. Zolnikova. Tetrahedron Lett., 44, 2003, 5453.
M. Takahashi and D. Suga. Synthesis, 1998, 986.
M. De Angelis, Fabio Stossi, K. A. Carlson, B. S. Katzenellenbogen, and J. A. Katzenellenbogen, J. Med. Chem., 2005, 48, 1132.
   The invention also provides a method for preparing a compound of formula (I) in accordance with the invention as described above wherein G is the following group:

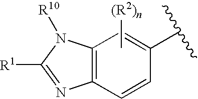

comprising a step of reacting
a compound of formula (IX)

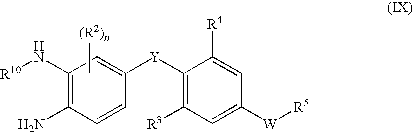

wherein R¹⁰, R², R³, R⁴, R⁵, Y and W are as defined above
with a compound of formula (X)

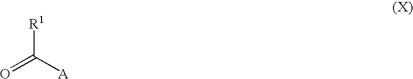

wherein R¹ is as defined above and A is H, OH, Cl or OCOR group where R is a C₁₋₄ alkyl group in the presence of a suitable acid, followed optionally by interconversion to another compound of formula (I).

Suitable acids for use in the reaction include sodium bisulphite.

Other acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Other suitable conditions and reagents suitable for use in the above reactions for the preparation of compounds of formula (I) in accordance with the invention or for the synthesis of intermediates suitable for preparing compounds of formula (I) are described in the following references:
Griffin, R. J. Et al.; Bioorg. Med. Chem. Lett.; 14; 10; 2004; 2433.
Uzunoglu, S; Tosun, A. U.; Oezden, T.; Yesilada; E.; Berkem, R.; Farmaco; 52; 10; 1997; 619.
Goeker, H.; Kus, C.; Boykin, D. W.; Yildiz, S.; Altanlar, N.; Bioorg. Sied Chem.; 10; 8; 2002; 2589.
Singh, M. P.; Sasmal, S.; Lu, W.; Chatterjee; M. N.; Synthesis; 10; 2000; 1380.
Beaulieu, C. et al; Bioorg. Med. Chem.; 14; 12; 2004; 3195.
Lee, In-Sook Han; Jeoung, E. H; Kreevoy, M.; J. Am. Chem. Soc., 119; 11; 1997; 2722.
Baudy, R. B.; Abou-Gharbia, M.; J Med. Chem.; 44, 10; 2001; 1516.
   Preferred compounds of formula (IX) include:
3-[4-(4-Amino-3-methylamino-phenoxy)-3,5-dibromo-phenyl]-propionic acid methyl ester
3-[4-(4-Amino-3-isopropylamino-phenoxy)-3,5-dibromo-phenyl]-propionic acid methyl ester
3-[4-(4-Amino-3-methylamino-phenoxy)-3,5-dibromo-phenyl]-2-fluoro-propionic acid methyl ester
3-[4-(4-Amino-3-ethylamino-phenoxy)-3,5-dibromo-phenyl]-2-fluoro-propionic acid methyl ester
3-[4-(4-Amino-3-methylamino-phenoxy)-3,5-dibromo-phenyl]-propionic acid methyl ester
(E)-3-[4-(4-Amino-3-ethylamino-phenoxy)-3,5-dibromo-phenyl]-acrylic acid methyl ester
[4-(4-Amino-3-methylamino-phenoxy)-3,5-dibromo-phenoxy]-acetic acid methyl ester
[4-(4-Amino-3-methylamino-phenoxy)-3,5-dibromo-benzoylamino]-acetic acid methyl ester
N-[4-(4-Amino-3-methylamino-phenoxy)-3,5-dibromo-phenyl]-malonamic acid methyl ester
N-[4-(4-Amino-3-ethylamino-phenoxy)-3,5-dibromo-phenyl]-malonamic acid methyl ester
   Preferred compounds of formula (X) include:
2-Methyl-propionaldehyde
4-Methyl-benzaldehyde
4-Bromo-benzaldehyde
3-Methoxy-benzaldehyde
   The invention also provides a method for preparing a compound of formula (I) in accordance with the invention as described above wherein G is the following group:

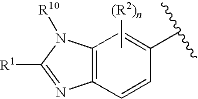

comprising a step of reacting
a compound of formula (XI)

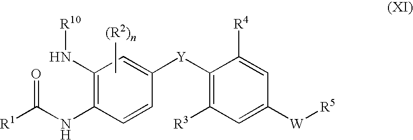

wherein R¹, R¹⁰, R², R³, R⁴, R⁵, Y and W are as defined above
in the presence of a suitable acid, followed optionally by interconversion to another compound of formula (I).

Suitable acids for use in the reaction include acetic acid.

Other acids may be employed, as is known by the person skilled in the art. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (XI) include:
Ethyl (3,5-dibromo-4-{3-(isopropylamino)₄-[(3-methylbutanoyl)amino]phenoxy}-phenoxy)acetate

Compounds of formula (XI) as described above may be prepared from a starting material of formula (V)

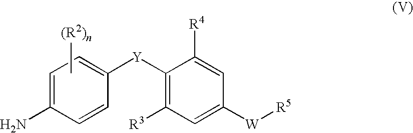

by reaction of the compound of formula (V)
with a compound of formula (XII)

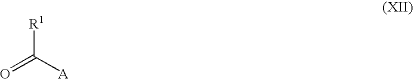

wherein R¹ is as defined above and A is a suitable leaving group, for example Cl, in the presence of a suitable base, for example an organic amine such as pyridine,
followed by introduction of the amino group, for example by nitration and subsequent reduction using suitable reagents, followed by installation of the R¹⁰ group onto the amino group.

For each step, the reaction mixture is stirred at room temperature, or heated until the starting materials have been consumed. Any step may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (V) include:
Methyl[4-(4-aminophenoxy)-3,5-dibromophenoxy]acetate)

Preferred compounds of formula (XII) include:
3-Methyl-butyryl chloride

The invention also provides a method for preparing a compound of formula (I) in accordance with the invention as described above wherein Y is methylene, comprising a step of reacting
a compound of formula (XIII)

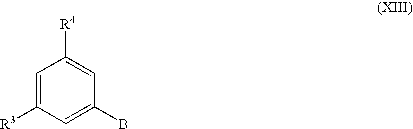

wherein R³ and R⁴, are as defined above and B is a group suitable for interconversion to the group —W—R⁵
with a compound of formula (XIV)

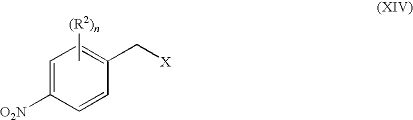

wherein R² is as defined above and X is a suitable leaving group, in the presence of a suitable base, followed by conversion of the group B to the group —W—R⁵, and reduction of the nitro group to an amino group using a suitable reducing agent, followed by interconversion to a compound of formula (I).

Suitable leaving groups X include halogen, for example a chloride. Suitable bases include lithium diisopropylamide or t-butyl lithium. Other combinations of leaving groups and bases may be employed, as is known by the person skilled in the art. Optionally, one or more coupling reagents may be employed. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Suitable groups B include alkyl groups, for example methyl. Conversion of the group B to the group —W—R⁵, may be achieved by the use of one or more suitable functional group interconversion reactions as known to the person skilled in the art.

Suitable reducing agents include hydrogen and a platinum oxide catalyst, iron in a suitable acid, for example hydrochloric acid, or SnCl₂ in ethanol. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Preferred compounds of formula (XIII) include:
1,3-Dibromo-5-methyl-benzene
   Preferred compounds of formula (XIV) include:
p-Nitro benzylchloride

The invention also provides a method for preparing a compound of formula (I) in accordance with the invention as described above wherein Y is selected from oxygen, sulphur or —N(R^{a})—, comprising a step of reacting
a compound of formula (II)

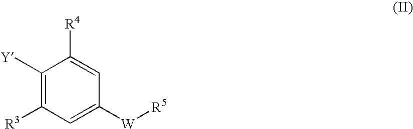

wherein W. R³, R⁴, and R⁵ are as defined above and Y′ is OH, SH or NR^{a}H
with a compound of formula (XV)

G-Z (XV)

wherein G is a group selected for example from:

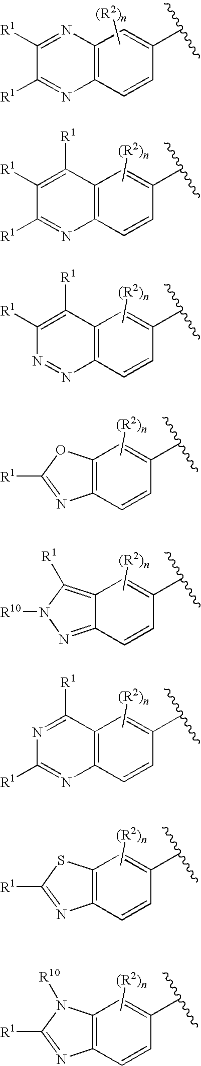

and wherein R¹, R¹⁰, R² and n are as defined above and Z is a suitable leaving group, optionally in the presence of a suitable base and optionally, in the presence of copper powder, followed optionally by removal of the protecting group, if present, and optionally by interconversion to another compound of the invention.

Suitable leaving groups Z include halogens and boron derivatives, for example a fluoride. Suitable bases include carbonates, alkylamines and alkali metal hydroxides, for example potassium carbonate, cesium carbonate, potassium hydroxide, sodium hydroxide, diisopropylamine and triethylamine. Other combinations of leaving groups and bases may be employed, as is known by the person skilled in the art. Optionally, one or more coupling reagents may be employed. The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups Y′ and Z could be switched, being the leaving group in the (11) fragment (the nucleophilic substituent, Z) and the electrophilic radical Y′ in the (XV) fragment.

Preferred compounds of formula (II) include:
Methyl 3-(3,5-Dibromo-4-hydroxy-phenyl)-propanoate
Methyl(E)-3-(3,5-Dibromo-4-hydroxy-phenyl)-acrylate
Methyl (3,5-Dibromo-4-hydroxy-phenoxy)-acetate
Methyl 3-(3,5-Dibromo-4-hydroxy-phenyl)-2-fluoro-propanoate
Methyl (3,5-Dibromo-4-hydroxy-benzoylamino)acetate

The invention also provides a method for preparing a compound of formula (I) in accordance with the invention as described above wherein Y is methylene, comprising a step of reacting
a compound of formula (XVI)

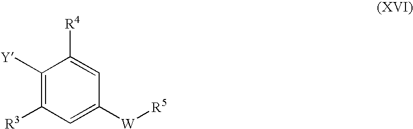

wherein W, R³, R⁴, and R⁵ are as defined above and Y′ is CHO
with a compound of formula (XVII)

G-Z (XVII)

wherein G is a group selected for example from:

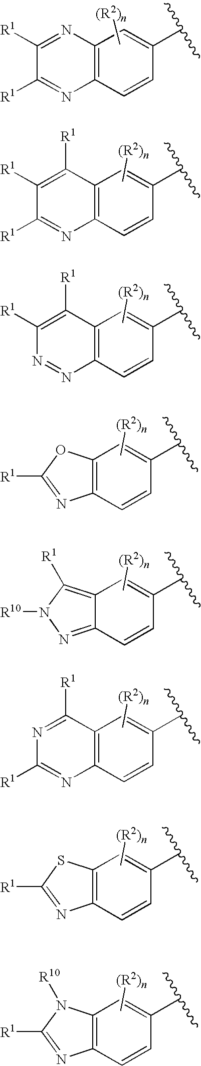

and wherein R¹, R¹⁰, R² and n are as defined above and Z may for example be lithium or a Mg-halide, such as MgBr or MgCl. Alternatively, Z may be a derivative of Sn, Pd, B or Cu.

Other combinations to produce a nucleophilic attack to an aldehyde may be employed, as is known by the person skilled in the art. Optionally, one or more coupling reagents may be employed. The reaction mixture may be stirred at room temperature or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The groups Y′ and Z could be switched, being the leaving group in the (XVI) fragment (the metal substituent, Z) and the aldehyde in the (XVII) fragment.

### EXAMPLES

The following compounds illustrate compounds of the invention or, where appropriate, compounds for use in the invention.

### General Experimental Conditions

Compounds were analyzed on HPLC-MS with alternating +/−API and equipped with different brands of 50 mm*2.1 mm, 5μ C8 columns. Elution was with 0.05% formic acid/acetonitrile or 0.05% ammonium acetate/acetonitrile.

MW calculated is an isotopic average and the “found mass” is referring to the most abundant isotope detected in the LC-MS.

### Intermediate 1

### Methyl 3-[4-(4-aminophenoxy)-3,5-dibromophenyl]propanoate

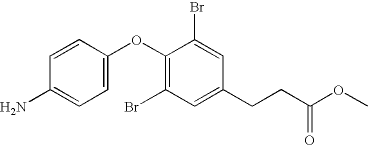

A solution of p-fluoro nitrobenzene (210 mg, 1.5 mmol), methyl 3-(4-hydroxy-3,5-dibromophenyl) propanoate (500 mg, 1.5 mmol) and potassium carbonate (410 mg, 3 mmol) in dimethylsulfoxide (3 ml) was purged with nitrogen and heated at 130° C. for 17 h. The mixture was diluted with ethyl acetate and washed with sodium bicarbonate (sat), water and brine. The combined organic phases were evaporated on silica and purified by flash chromatography (heptane/ethyl acetate 10:0 to 5:5) to give methyl 3-[3,5-dibromo-4-(4-nitrophenoxy)phenyl]propanoate as a white solid (504 mg, yield: 74%).

To a stirred solution of methyl 3-[3,5-dibromo-4-(4-nitrophenoxy)phenyl]propanoate (505 mg, 1.1 mmol) in acetic acid (25 ml) and water (3 ml), iron powder (308 mg, 5.5 mmol) was added. The reaction mixture was stirred for 17 h at 20° C. Acetic acid was removed under vacuum and the residue was diluted with ethyl acetate (50 mL) and water (50 mL) and extracted with ethyl acetate (2×5 mL). The combined ethyl acetate layers were washed with brine, dried over sodium sulphate and concentrated. The residue was purified by flash chromatography (dichloromethane/methanol 10:0 to 9:1) to afford the title compound (310 mg) in 72% yield. LC/MS (ESI) M+1_{found}=430.4 (MW_{calc}=429.1).

### Intermediate 2

### Methyl 4-[4-(4-aminophenoxy)-3,5-dibromophenyl]butanoate

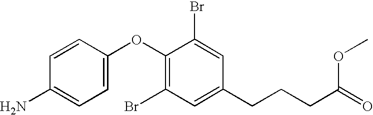

A solution of p-fluoro-nitrobenzene (282 mg, 2 mmol), methyl 3-(4-hydroxy-3,5-dibromophenyl) butanoate (704 mg, 2 mmol) and potassium carbonate (506 mg, 4 mmol) in dimethylsulfoxide (3 ml) was purged with nitrogen and heated to 130° C. for 17 h. The mixture was diluted with ethyl acetate and washed with sodium bicarbonate (sat), water and brine. The combined organic phases were evaporated on silica and purified by flash chromatography (heptane/ethyl acetate 10:0 to 5:5) to give methyl 3-[3,5-dibromo-4-(4-nitrophenoxy)phenyl]butanoate as a white solid (541 mg, 57%).

Methyl 3-[3,5-dibromo-4-(4-nitrophenoxy)phenyl]butanoate was dissolved in acetic acid (18 ml) and water (2 ml), and iron powder (310 mg, 5 eq) was added. The reaction mixture was stirred at room temperature under nitrogen for 17 h. The solvents were evaporated under vacuum and the residue was partionated between water and ethyl acetate. The water phase was extracted with ethyl acetate (2×10 mL) and the combined organic phases were washed with brine and dried over sodium sulphate. After evaporation, the crude was purified by flash chromatography (dichloromethane/methanol 10:0 to 9:1). Evaporation gave the title compound (310 mg, 64%) as a white solid.

### Intermediate 3

### Ethyl[4-(4-aminophenoxy)-3,5-dibromophenoxy]acetate

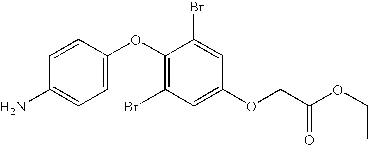

Sodium methoxide (2.2 g, 40 mmol) was added to a solution of 1,3-dibromo-5-fluoro-2-(4-nitrophenoxy)benzene (4 g, 10 mmol) in dimethylformamide (15 mL) at room temperature. The mixture was stirred at room temperature for 4 h. Water (20 mL) was added to the mixture and the product was extracted with ethyl acetate. The combined organic phases were washed consecutively with diluted hydrochloric acid and brine, dried over anhydrous magnesium sulphate and concentrated in vacuo. This crude mixture was used immediately without further purification. Boron trifluoride-methyl sulfide complex (1M, 12.8 mL, 12.8 mmol) was added dropwise to a stirred, chilled (dry ice-acetone bath) solution of crude 1,3-dibromo-5-methoxy-2-(4-nitrophenoxy)benzene (4.9 g, 12 mmol) in dichloromethane (150 mL). The mixture was allowed to warm up to room temperature and was stirred overnight. The reaction mixture was concentrated under vacuum, diluted with water, and extracted with ethyl acetate. The combined organic phases were washed with diluted hydrochloric acid, saturated sodium bicarbonate and brine, dried over anhydrous magnesium sulphate and concentrated in vacuo. The residue was purified by flash chromatography (petroleum ether/ethyl acetate 20:1) to give 2.5 g (64.3%) of 3,5-dibromo-4-(4-nitrophenoxy)phenol as light yellow oil.

Ethyl bromoacetate (2.5 mL, 22 mmol) was added to a mixture of 3,5-dibromo-4-(4-nitrophenoxy)phenol (5.2 g, 13 mmol) and potassium carbonate (7.6 g, 54 mmol) in acetone (150 mL) at 0° C. After being stirred at ambient temperature for 4 h, the mixture was concentrated in vacuo. Ethyl acetate was added to the residue and the organic phase was washed with brine, dried over anhydrous magnesium sulphate and concentrated in vacuo to give the crude mixture of ethyl[3,5-dibromo-4-(4-nitrophenoxy)phenoxy]acetate which was used without further purification. To a solution of ethyl[3,5-dibromo-4-(4-nitrophenoxy)phenoxy]acetate (3.8 g, 8 mmol) in ethanol (150 mL), tin(II) chloride (9 g, 47 mmol) was added and the reaction mixture was stirred overnight at 80° C. After cooling to room temperature, the mixture was concentrated in vacuo and ethyl acetate and water were added to the residue. The organic phase was washed with sodium hydroxide (25% aqueous) and brine, dried over anhydrous potassium carbonate and concentrated in vacuo. The residue was purified by flash chromatography (petroleum ether/ethyl acetate 4:1 to 2:1) to give 1.2 g (64.3%) of ethyl[4-(4-aminophenoxy)-3,5-dibromophenoxy]acetate. LC/MS (ESI) M+1_{found}=446.3 (MW_{calc}=445.1).

### Intermediate 4

### Ethyl (2E)-3-[4-(4-aminophenoxy)-3,5-dibromophenyl]acrylate

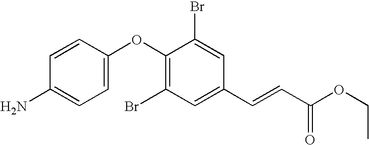

A mixture of 3,5-dibromo-4-(4-nitrophenoxy)phenol (2.5 g, 6.4 mmol) and triethylamine (1.2 mL, 8.6 mmol) in dichloromethane (80 mL) was added dropwise to a 0° C. solution of trifluoromethanesulfonic anhydride (1.4 mL, 8.3 mmol) in dichloromethane (20 mL). The mixture was allowed to reach room temperature and stirred overnight. The reaction mixture was washed with water and dried over anhydrous magnesium sulphate, filtered and concentrated in vacuo. The obtained residue containing 3,5-dibromo-4-(4-nitrophenoxy)phenyl trifluoromethanesulfonate was used without further purification.

A mixture of 3,5-dibromo-4-(4-nitrophenoxy)phenyl trifluoromethanesulfonate (340 mg, 0.6 mmol), ethyl acrylate (85 μL, 0.8 mmol), N-Ethyl diisopropylamine (129 μL, 0.7 mmol) and bis(triphenylphosphine)palladium(II) chloride (23 mg) in dimethylformamide (5 mL) was placed in a Parr bomb and heated at 110° C. overnight. After cooling to room temperature, the mixture was diluted with ethyl acetate and filtered. The filtrate was washed with water, dried over anhydrous magnesium sulphate, filtered and concentrated in vacuo. The residue was purified by flash chromatography (petroleum ether/ethyl acetate 9.5:0.5) to give 129 mg of ethyl (2E)-3-[4-(4-nitrophenoxy)-3,5-dibromophenyl]acrylate.

To a solution of ethyl (2E)-3-[4-(4-nitrophenoxy)-3,5-dibromophenyl]acrylate (0.5 g, 1 mmol) in ethanol (100 mL), tin(II) chloride (2.5 g, 13 mmol) was added and the reaction mixture was stirred overnight at 80° C. After cooling to ambient temperature, the mixture was concentrated in vacuo, ethyl acetate and water were added. The organic phase was washed with sodium hydroxide (25% aqueous) and brine, dried over anhydrous potassium carbonate and concentrated in vacuo. The resulting residue was purified by flash chromatography (petroleum ether/ethyl acetate 4:1 to 2:1) to give 210 mg of ethyl (2E-3-[4-(4-aminophenoxy)-3,5-dibromophenyl]acrylate.

LC/MS (ESI) M_{found}=441.9 (MW_{calc}=441.1).

### Intermediate 5

### Methyl N-[4-(4-aminophenoxy)-3,5-dibromobenzoyl]glycinate

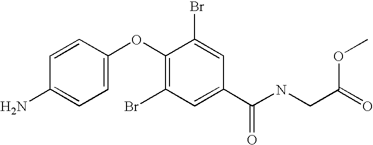

Bromine (5.75 ml, 54.6 mmol) in glacial acetic acid (80 mL) was added dropwise into a solution of p-cresol (5.9 g, 54.6 mmol) in acetic acid (12 mL) and water (33 mL) in a water bath. The reaction solution was stirred for additional 0.5 h at room temperature and poured into water (200 mL). The precipitate was collected and purified by recrystallisation from ethyl acetate/petroleum ether. 2,6-Dibromo-4-methyl-phenol (13.2 g) was obtained in 91% yield.

Sodium hydride (0.46 g, 13.4 mmol) was pre-washed with hexane (to remove coal oil/kerosene) and was carefully dissolved in methanol (41 mL, anhydrous). 2,6-Dibromo-4-methyl-phenol (3.43 g, 12 mmol) was added to the basic solution. The solvent was evaporated to obtain a white solid which was mixed with dimethylsulfoxide (18.5 mL, anhydrous) and p-dinitrobenzene (1.90 g, 11.3 mmol), and heated at 90° C. for 16 h (water free conditions). The reaction mixture was poured into 500 mL of water/ice, and extracted with diethyl ether (3×500 mL). The combined organic phases were washed with aqueous sodium hydroxide (5%, 200 mL) and water (200 mL), dried and concentrated. The residue was purified by flash chromatography to give 1,3-Dibromo-5-methyl-2-(4-nitro-phenoxy)benzene (2.6 g) in 59% of yield.

1,3-Dibromo-5-methyl-2-(4-nitro-phenoxy)-benzene (2.60 g, 6.7 mmol) was dissolved in pyridine (30 mL) and water (12 mL), and heated up to refluxing temperature. Potassium pernanganate (8.5 g, 53.8 mmol) was added in portions to the refluxing solution and the mixture was allowed to cool down and stirred for 6 h at room temperature. The reaction solution was diluted with ethyl acetate and filtered with celite. The residue obtained after evaporation of the solvent was diluted with hydrochloric acid (2M) and extracted with ethyl acetate. The combined organic phases were washed with sodium hydroxide (5%, aqueous). The alkaline solution was acidified with hydrochloric acid and extracted with acetate. The combined ethyl acetate phases were dried and concentrated to give 3,5-dibromo-4-(4-nitro-phenoxy)-benzoic acid (2.0 g) in 71% yield.

3,5-Dibromo-4-(4-nitro-phenoxy)-benzoic acid (1.6 g, 3.85 mmol), glycine methyl ester (hydrochloride salt, 1.54 g, 4.17 mmol), 3-ethyl-1-[3-(dimethylamino)propyl]carbodiimide hydrochloride (EDCI) (3.22 g, 5.77 mmol), 1-hydroxybenzotriazole hydrate (HOBt) (2.27 g, 5.76 mmol) were dissolved in anhydrous dichlorometane (50 mL). After the addition of triethylamine (1.6 mL 11.5 mmol), the reaction mixture was stirred overnight at room temperature. Direct purification by flash chromatography (or pre-washed with water) gave methyl N-[4-(4-nitrophenoxy)-3,5-dibromobenzoyl]glycinate (1.4 g) in a 75% yield

Methyl N-[4-(4-nitrophenoxy)-3,5-dibromobenzoyl]glycinate (1.45 g, 3 mmol) and platinum oxide (72.5 mg, 0.3 mmol.) were suspended in ethyl acetate (120 mL), and hydrogenated under normal pressure for 30 h. The reaction solution was filtered, concentrated and purified by flash chromatography. Methyl N-[4-(4-aminophenoxy)-3,5-dibromobenzoyl]glycinate (1.0 g) was obtained in 75% yield.

LC/MS (ESI) M_{found}=458.7 (MW_{calc}=458.1).

### Preparation of Quinolines

### General Procedure A for the Preparation of Examples 1-25

To a refluxing solution of the appropriate aniline (1.0 equiv) (e.g. methyl 3-[4-(4-aminophenoxy) 3,5-dibromophenyl]propanoate) in hydrochloric acid (6 N, 2 mL/mmol) was added dropwise the appropriate aldehydelketone (e.g (E)-Hept-3-en-2-one) (1.2 equiv) in dioxane (1 mL/mmol). The resulting mixture was heated under reflux for 2 h in a closed valve. After cooling to room temperature, the mixture was eluted through a C-18 SPE-column (Isolute 0.5 g) using first water then methanol. The crude product was purified using semi-preparative-HPLC (Zorbax CombiHT (SB-C8) Mobil Phase: Solvent A. Water with 0.5% formic acid; 5 Solvent B: acetonitrile. Gradient: 80% of A to 5% of A) to yield the desired quinoline (e.g. 3-{3,5-dibromo-4-[(4-methyl-2-propylquinolin-6-yl)oxy]phenyl}propanoic acid).

### Preparation of Examples 26-28

Malonic acid (5 mg, 0.05 mmol) and ethyl[4-(4-aminophenoxy)-3,5-dibromophenoxy]acetate (22 mg, 0.05 mmol) were dissolved in phosphorus oxychloride (100 μL). The mixture was boiled under gentle reflux for 3 h. The cooled mixture was eluted through a C-18 SPE-column (Isolute 0.5 g) using first water then methanol.

The crude was dissolved in methanol (2 mL) and sodium methoxide (100 mg) was added. The mixture was stirred at room temperature overnight. The crude product was purified using semi-preparative-HPLC (Zorbax CombiHT (SB-C8) Mobil Phase: Solvent A. Water with 0.5% formic acid; 5 Solvent B: acetonitrile. Gradient: 80% of A to 5% of A). Three compounds were isolated, {3,5-dibromo-4-[(2,4-dichloroquinolin-6-yl)oxy]phenoxy}acetic acid, {3,5-dibromo-4-[(4-chloro-2-hydroxyquinolin-6-yl)oxy]phenoxy}acetic acid and {3,5-dibromo-4-[(2,4-dihydroxyquinolin-6-yl)oxy]phenoxy}acetic acid.

### Preparation of Examples 29-30

Ethyl{3,5-dibromo-4-[(2,4-dichloroquinolin-6-yl)oxy]phenoxy}acetate was dissolved in methanol (0.5 mL) and sodium methoxide (100 mg) was added. The mixture was stirred at reflux overnight in a closed valve. The crude product was purified using semi-preparative-HPLC (Zorbax CombiHT (SB-C8) Mobil Phase: Solvent A. Water with 0.5% formic acid; 5 Solvent B: acetonitrile. Gradient: 80% of A to 5% of A) to yield {3,5-dibromo-4-[(2,4-dimethoxyquinolin-6-yl)oxy]phenoxy}acetic acid and {3,5-dibromo-4-[(4-chloro-2-methoxyquinolin-6-yl)oxy]phenoxy}acetic acid.

### General Procedure B for the Preparation of Examples 31-33

A stirred mixture of potassium carbonate (3 equiv.), the appropriate quinoline (1 equiv.) (e.g. 6-hydroxy-quinoline) (commercially available or prepared from para aminophenol), and the appropriate iodo-benzene (e.g. 3,5-dibromo-4-iodo-nitrobenzene) (1 equiv.) in dimethylformamide (14 mL/mmol) was heated for 18 h at 70° C. After dilution with diethyl ether and ammonium chloride (saturated aq. solution), the mixture was extracted with diethyl ether. The combined organic layers were washed with ammonium chloride (saturated aq. solution) followed by drying over magnesium sulphate. After removal of the volatiles, chromatography on silica gel with dichloromethane/diethyl ether (1:1) eluted the desired biphenylether (e.g. 6-(2,6-Dibromo-4-nitro-phenoxy)-quinoline).

The biphenylether (e.g. 6-(2,6-Dibromo-4-nitro-phenoxy)-quinoline) (1 equiv.) and tin(II) chloride (5 equiv.) were dissolved in ethanol (40 mL/mmol) and stirred at reflux for 3 h. Ethyl acetate and sodium carbonate (sat. aq. solution) were added to the reaction. The organic phase was separated and dried (magnesium sulphate). The crude was dissolved in tetrahydrofuran (20 mL/mmol) and triethylamine (2.5 equiv.) was added, followed by ethyl malonyl chloride (1.5 equiv.). The reaction was stirred overnight at room temperature. Ammonium chloride (sat. aq. solution) was added and the product was extracted into ethyl acetate and dried using magnesium sulphate. The solvent was evaporated and the remaining residue was redissolved in dioxane (8 mL/mmol) and treated with potassium hydroxide (25 mL/mmol, 2M). The reaction was stirred for 3 h and purified by semi-preparative HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) which gave the wanted acid (e.g. 3-{[3,5-dibromo-4-(quinolin-6-yloxy)phenyl]amino}-3-oxopropanoic acid).

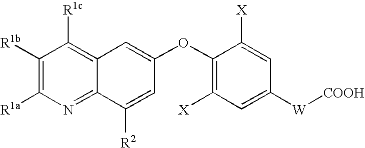

| | | | | | | | Yield | | M + 1 |
|---|---|---|---|---|---|---|---|---|---|
| Example | R^{1a} | R^{1b} | R^{1c} | R² | X | W | (%) | MW (calc) | (found) |
| 1 | Me | H | H | H | Cl | (CH₂) | 8 | 362.2 | 362.1 |
| 2 | Me | H | H | H | Br | (CH₂)₂ | 20 | 465.1 | 466.1 |
| 3 | Me | H | Me | H | Br | (CH₂)₂ | 21 | 479.2 | 480.1 |
| 4 | Et | H | H | H | Br | (CH₂)₂ | 14 | 479.2 | 478.1 |
| | | | | | | | | | (M − 1) |
| 5 | Pr | H | H | H | Br | (CH₂)₂ | 14 | 493.2 | 494.3 |
| 6 | Pr | H | Me | H | Br | (CH₂)₂ | 41 | 507.2 | 508.1 |
| 7 | 1- | H | H | H | Br | (CH₂)₂ | 2 | 549.3 | 550.4 |
| | Hept | | | | | | | | |
| 8 | Me | H | Et | H | Br | (CH₂)₂ | 13 | 493.2 | 494.0 |
| 9 | Me | H | Bu | H | Br | (CH₂)₂ | 21 | 521.3 | 522.1 |
| 10 | Pr | Et | H | H | Br | (CH₂)₂ | 2 | 521.3 | 522.1 |
| 11 | Pr | H | Me | Cl | Br | (CH₂)₂ | 6 | 541.7 | 544.1 |
| | | | | | | | | | (M + 2) |
| 12 | Me | H | Me | H | Br | OCH₂ | 3 | 481.1 | 482.0 |
| 13 | Me | H | H | H | Br | OCH₂ | 46 | 467.1 | 468.0 |
| 14 | Pr | H | Me | H | Br | OCH₂ | 5 | 509.2 | 510.1 |
| 15 | Et | H | H | H | Br | OCH₂ | 6 | 481.1 | 482.0 |
| 16 | Me | H | Me | H | Br | CH═CH | 8 | 477.2 | 478.0 |
| 17 | Pr | H | Me | H | Br | CH═CH | 8 | 505.2 | 506.0 |
| 18 | Me | H | Me | H | Cl | (CH₂)₂ | 14 | 390.3 | 390.1 |
| | | | | | | | | | (M) |
| 19 | Pr | H | Me | H | Cl | (CH₂)₂ | 11 | 418.3 | 418.1 |
| | | | | | | | | | (M) |
| 20 | Pr | H | Me | Me | Br | (CH₂)₂ | 2 | 521.3 | 522.1 |
| 21 | Pr | H | Me | OH | Br | (CH₂)₂ | 3 | 523.2 | 524.1 |
| 22 | Pr | H | Me | H | Br | — | 4 | 479.2 | 480.0 |
| 23 | Pr | H | Me | H | Br | CONHCH₂ | 3 | 536.2 | 537.0 |
| 24 | Pr | H | Me | CF₃ | Br | (CH₂)₂ | 3 | 575.3 | 576.1 |
| 25 | Pr | H | Me | H | Br | CH₂CHF | 8 | 525.2 | 526.4 |
| 26 | Cl | H | Cl | H | Br | OCH₂ | 6 | 522.0 | 521.9 |
| | | | | | | | | | (M) |
| 27 | Cl | H | OH | H | Br | OCH₂ | 5 | 503.5 | 504.0 |
| 28 | OH | H | OH | H | Br | OCH₂ | 7 | 485.1 | 486.0 |
| 29 | OMe | H | OMe | H | Br | OCH₂ | 3 | 513.1 | 514.0 |
| 30 | Cl | H | OMe | H | Br | OCH₂ | 2 | 517.6 | 517.9 |
| | | | | | | | | | (M) |
| 31 | H | H | H | H | Br | NHCOCH₂ | 4 | 480.1 | 481.0 |
| 32 | H | H | Me | H | Br | NHCOCH₂ | 5 | 494.1 | 495.0 |
| 33 | Pr | H | Me | H | Br | NHCOCH₂ | 17 | 536.2 | 537.1 |

### Example 34

### ({3,5-dibromo-4-[(4-methyl-2-propylquinolin-6-yl)methyl]benzyl}oxy)acetic acid

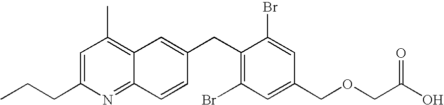

To a solution of 1,3-Dibromo-5-methyl-benzene (0.25 g, 1 mmol) in tetrahydrofuran (2 mL) at −78° C. was added lithium diisopropylamide (1.2 mL, 1.2 mmol, IM) the mixture was stirred for 30 min. p-Nitro benzylchloride (0.26 g, 1.2 mmol) in tetrahydrofuran (1 mL) was added. The reaction was stirred for 16 h and allowed to reach room temperature. Water and diethyl ether were added and the mixture was extracted with diethyl ether (3×10 mL). The organic phases were collected and dried. The solvents were distilled off and the product purified by flash chromatography (diethyl ether/heptane 1:3) to give 0.15 g (40% yield) of pure 1,3-dibromo-5-methyl-2-(4-nitrobenzyl)benzene.

N-bromosuccinimide (23 mg, 0.13 mmol) was added to the mixture of 1,3-dibromo-5-methyl-2-(4-nitro-benzyl)-benzene (50 mg, 0.13 mmol) in tetrachloromethane (2 mL). The mixture was stirred at reflux for 1 h. Filtration through silica with dichloromethane followed by evaporation of the solvents gave a crude product (1,3-dibromo-5-bromomethyl-2-(4-nitro-benzyl)-benzene) which was dissolved in dioxane (3 mL) and potassium hydroxide (6 mL, aq. 2M) was added. The mixture was refluxed overnight. Extraction (dichloromethane phase separator) and evaporation gave 33 mg of a dry crude product ([3,5-dibromo-4-(4-nitro-benzyl)-phenyl]-methanol) which was used without further purification, 62% crude yield.

To the solution of [3,5-dibromo-4-(4-nitro-benzyl)-phenyl]-methanol (33 mg, 0.08 mmol) in tetrahydrofuran (1 mL), was added sodium hydride (6.4 mg, 0.16 mmol). The mixture was stirred for 5 min. Teri-butyl bromoacetate (23 μL, 0.16 mmol) was added and the reaction was stirred for 15 h. Ethyl acetate and water were added and the product was extracted, dried and evaporated. The residue was dissolved in ethanol (3 mL) and tin(II) chloride (0.1 g, 0.4 mmol) was added. The mixture was stirred at reflux for 2 h. Ethyl acetate and saturated aqueous solution of sodium carbonate were added and the product was extracted, dried and evaporated. The product was purified by flash chromatography (dichloromethane) to give 10 mg of [4-(4-Amino-benzyl)-3,5-dibromo-benzyloxy]-acetic acid tert-butyl ester, 25% yield.

[4-(4-Aminobenzyl)-3,5-dibromo-benzyloxy]-acetic acid tert-butyl ester was transformed into ({3,5-dibromo-4-[(4-methyl-2-propylquinolin-6-yl)methyl]benzyl}oxy)acetic acid applying the general method previously described for the preparation of the quinoline, giving 0.61 mg of the quinoline (6% yield).

LC-MS (ESI) M+1_{found}=522.1 (MW_{calc}=521.2)

### Example 35

### 3-{[3,5dibromo-4-({2-[(methylamino)carbonyl]quinolin-6-yl}oxy)phenyl]amino}-3-oxopropanoic acid

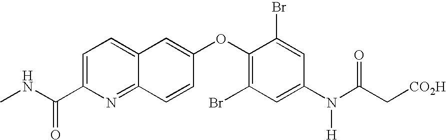

A 4-mL vial equipped with a stir bar was oven dried. 5-hydroxy-2-nitrobenzaldehyde (0.076 g, 0.45 mmol) and ethyl pyruvate (0.053 g, 0.45 mmol) were added, followed by anhydrous ethanol (2.7 mL). Tin(II) chloride (dry) (0.43 g, 2.3 mmol, 5 equiv), zinc chloride (0.31 g, 2.3 mmol, 5 equiv), and 4 Å molecular sieves (approximately 0.1 g) were added to the solution. This mixture was then heated at 70° C. under an atmosphere of nitrogen for 3 h. The reaction was then cooled to room temperature. The mixture was transferred to a separatory funnel using ethyl acetate which was washed with water, dried over magnesium sulphate, filtered and concentrated. The obtained residue was purified by chromatography on silica column (gradient ethyl acetate/heptane). The desired ethyl 6-hydroxy-quinoline-2-carboxylate was obtained in 27% yield (26 mg).

To ethyl 6-hydroxy-quinoline-2-carboxylate (26 mg, 0.12 mmol) was added methylamine (4 mL, 2M) in methanol. The reaction was stirred at room temperature over night. Additional methylamine (2 mL, 2M) in methanol was added and the reaction was once again stirred over night. The mixture was concentrated and purified by flash chromatography (methanol/dichloromethane 1:9) to give methyl 6-hydroxy-quinoline-2-carboxylamide (14 mg, 58%).

Methyl 6-hydroxy-quinoline-2-carboxylamide (14 mg, 0.07 mmol) was dissolved in dimethylformamide (1 mL). 1,3-dibromo-2-iodo-5-nitrobenzene (28 mg, 0.07 mmol) and potassium carbonate (30 mg, 0.022 mmol) were added. The reaction was stirred at 75° C. over night. Diethyl ether was added and the organic phase was washed with ammonium chloride (saturated aqueous solution), dried over magnesium sulphate and concentrated to give 43 mg of a crude 6-(2,6-dibromo-4-nitrophenoxy)-N-methylquinoline-2-carboxamide which was used as such.

The crude 6-(2,6-dibromo-4-nitrophenoxy)-N-methylquinoline-2-carboxamide was dissolved in ethanol (3 mL) and tin(II) chloride hydrate (90 mg, 0.4 mmol) was added. The reaction was refluxed for 3 h. The reaction mixture was then cooled to room temperature and transferred to a separatory funnel using ethyl acetate which was washed with sodium carbonate (saturated aqueous solution), dried over magnesium sulphate, filtered and concentrated. The remaining residue was purified by chromatography on silica (gradient diethyl ether/dichloromethane). The desired 6-(4-amino-2,6-dibromophenoxy)N-methylquinoline-2-carboxamide was obtained in 86% yield (29 mg) over two steps.

6-(4-Amino-2,6-dibromophenoxy)-N-methylquinoline-2-carboxamide (29 mg, 0.06 mmol) was dissolved in dry THF (1.2 mL), and triethylamine (21 μL, 0.15 mmol) and ethyl malonyl chloride (12 μL, 0.09 mmol) were added. The reaction was stirred over night at room temperature. The reaction was transferred to a separatory funnel using ethyl acetate which was washed with ammonium chloride (saturated aqueous solution), dried over magnesium sulphate, filtered and concentrated. The crude product was hydrolyzed by dissolving it in dioxane (2 mL) and treatment with potassium hydroxide (2M, 6 mL) and stirring for 2 h. Purification using semi-preparative-HPLC (Zorbax CombiHT (SB-C8 50×21.2 mm, 51) Mobile Phase: Solvent A. Water with 0.5% formic acid; Solvent B: acetonitrile. Gradient: 2 min 80% of A then over 8 min to 5% of A) gave the desired 3-{[3,5-dibromo-4-({2-[(methylamino)carbonyl]quinolin-6-yl}oxy)phenyl]amino}-3-oxopropanoic acid, obtained in 28% yield over two steps (9 mg).

LC-MS (ESI) M+1_{found}=538.4 (MW_{calc}=537.2)

### Example 36

### 3-({3,5dibromo-4-[(2-{[(methylsulfonyl)amino]methyl}quinolin-6-yl)oxy]phenyl}amino)-3-oxopropanoic acid

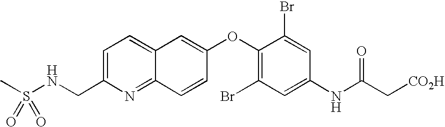

To a dimethylformamide (14 mL) solution of 2-methyl-quinolin-6-ol (160 mg, 1.01 mmol) were added 1,3-dibromo-2-iodo-5-nitrobenzene (410 mg, 1.01 mmol) and potassium carbonate (420 mg, 3.03 mmol). The reaction mixture was stirred at 75° C. over night. Diethyl ether was added and the organic phase was washed with ammonium chloride (saturated aqueous solution), dried over magnesium sulphate and concentrated. The obtained residue was purified by flash chromatography (ethyl ether/dichloromethane 1:9) to give 6-(2,6-dibromo-4-nitrophenoxy)-2-methylquinoline (300 mg, 68% yield).

6-(2,6-Dibromo-4-nitrophenoxy)-2-methylquinoline (300 mg, 0.68 mmol) was dissolved in carbon tetrachloride (10 mL). Benzoylperoxide (10 mg) and of N-bromosuccinimide (0.18 g, 1.03 mmol) were added. The reaction was refluxed over night and then concentrated to give a crude product which was purified by flash chromatography (ethyl ether/dichloromethane) to give 2-(bromomethyl) 6-(2,6-dibromo-4-nitrophenoxy)quinoline (142 mg, 40% yield).

2-(Bromomethyl)-6-(2,6-dibromo-4-nitrophenoxy)quinoline (120 mg, 0.23 mmol) in ammonia (5 mL, 7M in methanol) was heated in a microwave (automated EmryS™ Optimizer single mode microwave reactor from Biotage AB) (130° C., 15 min). The mixture was concentrated and purified by flash chromatography (methanol/dichloromethane 2:8) to give 1-[6-(2,6-dibromo-4-nitrophenoxy)quinolin-2-yl]methanamine (32 mg, 31% yield).

A solution of 1-[6-(2,6-dibromo-4-nitrophenoxy)quinolin-2-yl]methanamine (32 mg, 0.07 mmol) in dry dichloromethane (1 mL) was treated with methanesulfonyl chloride (5 μL, 0.07 mmol) and pyridine (14 μL, 0.18 mmol). The reaction mixture was stirred over night at room temperature. The mixture was concentrated and purified by flash chromatography (methanol/dichloromethane 5:95) to give N-{[6-(2,6-dibromo-4-nitrophenoxy)quinolin-2-yl]methyl}methanesulfonamide (22 mg, 58%).

The crude N-{[6-(2,6-dibromo-4-nitrophenoxy)quinolin-2-yl]methyl}methanesulfonamide (22 mg, 0.04 mmol) was dissolved in ethanol (1.6 mL) and tin(II) chloride hydrate (47 mg, 0.21 mmol) was added. The reaction mixture was refluxed for 2 h. The reaction was then cooled to room temperature and transferred to a separation funnel using ethyl acetate. The organic phase was washed with sodium carbonate (saturated aqueous solution), dried over magnesium sulphate, filtered and concentrated to give a crude N-{[6-(4-amino-2,6-dibromophenoxy)quinolin-2-yl]methyl}methanesulfonamide.

The crude N-{[6-(4-amino-2,6-dibromophenoxy)quinolin-2-yl]methyl}methanesulfonamide was dissolved in dry tetrahydrofuran (1.2 mL) and triethylamine (20 μL, 0.15 mmol) and ethyl malonyl chloride (12 μL, 0.09 mmol) were added. The reaction was stirred over night at room temperature. The reaction was transferred to a separatory funnel using ethyl acetate. The organic phase was washed with ammonium chloride (saturated aqueous solution) dried over magnesium sulphate, filtered and concentrated. The crude product was hydrolyzed by dissolving it in dioxane (2 mL) and treatment with potassium hydroxide (6 mL, 2M) and stirring for 2 h. Purification using semi-preparative-HPLC (Zorbax CombiHT (SB-C8 50×21.2 mm, 51) Mobile Phase: Solvent A. Water with 0.5% formic acid; Solvent B: acetonitrile. Gradient: 2 min 80% of A then over 8 min to 5% of A) gave 7 mg of the desired 3-({3,5-dibromo-4-[(2-{[(methylsulfonyl)amino]methyl}quinolin-6-yl)oxy]phenyl}amino)-3-oxopropanoic acid, obtained in 30% yield over three steps.

LC-MS (ESI) M+1_{found}=587.9 (MW_{calc}=587)

### Preparation of Benzothiazoles.

### General Procedure C for the Preparation of Examples 37-38

To a slurry of potassium ferrocyanide (3.8 equiv.) in water (1 mL/mmol) was added dropwise the appropriate thioamide (e.g. methyl 3-{3,5-dibromo-4-[4-(ethanethioylamino)phenoxy]phenyl}propanoate) in 10% sodium hydroxide in water (16 mL/mmol). The resulting mixture was stirred at room temperature for 16 h. The mixture was eluted through a C-18 SPE-column (Isolute 0.5 g) using first water then methanol, which contained the crude product. The crude product was purified using semi-preparative-HPLC (Zorbax CombiHT (SB-C8) Mobil Phase: Solvent A. Water with 0.5% formic acid; 5 Solvent B: acetonitrile. Gradient: 80% of A to 5% of A) to yield the desired benzothiazole (e.g. 3-{3,5-dibromo-4-[(2-methyl-1,3-benzothiazol-6-yl)oxy]phenyl}propanoic acid).

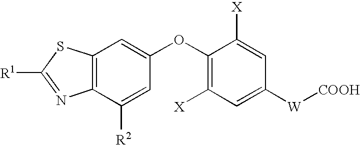

| | | | | | Yield | MW | M + 1 |
|---|---|---|---|---|---|---|---|
| Example | R¹ | R² | X | W | (%) | (calc) | (found) |
| 37 | Me | H | Br | (CH₂)₂ | 5 | 471.2 | 472.1 |
| 38 | Ph | H | Br | (CH₂)₂ | 5 | 533.2 | 534.0 |

### Preparation of Benzimidazoles.

### General Procedure D for the Preparation of Examples 39-46

To a dichloromethane solution of the appropriate aniline (e.g. methyl[4-(4-aminophenoxy)-3,5-dibromophenoxy]acetate) were added pyridine (10 mL/mmol) and the appropriate acid chloride (e.g. 3-methyl-butyryl chloride) (1.2 equiv.). The mixture was stirred at room temperature for 17 h. After acidification with hydrochloric acid (2M, aqueous solution), the product was extracted with chloroform using a phase separator. The solvents were evaporated and the last remains of pyridine were coevaporated with toluene. The obtained residue contained the wanted amide (e.g. methyl (3,5-dibromo-4-{4-[(3-methylbutanoyl)amino]phenoxy}phenoxy)acetate) and was used without further purification.

A solution of the appropriate amide (e.g. methyl (3,5-dibromo-4-{4-[(3-methylbutanoyl)amino]phenoxy}phenoxy)acetate) in acetic acid (40 mL/mmol) was cooled at 0° C. and sulphuric acid (10 mL/mmol) was added. After additional 10 minutes cooling, fuming nitric acid (1.8 equiv.) was added. The solution was stirred at 0° C. for 1 h and then at room temperature for 17 h. The reaction was quenched by addition of ice. The mixture was extracted with ethyl acetate and the combined organic layers were washed with brine and dried over sodium sulphate. The solvent was evaporated under vacuum to give the wanted nitro derivative (e.g. methyl (3,5-dibromo 4-{4-[(3-methylbutanoyl)amino]-3-nitrophenoxy}phenoxy)acetate) which could be used without further purification.

Platinum oxide (0.4 equiv.) was added to an ethyl acetate solution of the appropriate nitro derivative (e.g. ethyl (3,5-dibromo-4-{4-[(3-methylbutanoyl)amino]-3-nitrophenoxy}phenoxy)acetate) and the mixture was stirred under a 1.2 bar pressure of hydrogen for 17 h at 20° C. The residue was filtrated through a celite pad which was rinsed with ethyl acetate. Evaporation of the solvent gave the wanted aniline (e.g. methyl (4-{3-amino-4-[(3-methylbutanoyl)amino]phenoxy}-3,5-dibromophenoxy)acetate) which could be used without further purification.

Dimethylsulfide boran (1M, 6 equiv.) was added to a stirred solution of the appropriate aniline (e.g. methyl (4 {3-amino-4-[(3-methylbutanoyl)amino]phenoxy}-3,5-dibromophenoxy)acetate) (I equiv.) and acetic acid (2 ml/mmol) in dichloromethane (65 ml/mmol), dry acetone (16 mL/mmol) and tetrahydrofuran (65 ml/mmol) at 0° C. The reaction mixture was stirred for 17 h. Ethyl acetate and hydrochloric acid (2M) were added and the water phases were extracted with ethyl acetate (2×10 mL). The combined ethyl acetate phases were washed with brine and dried over sodium sulphate. The solvents were evaporated and the crude obtained mixture, containing the desired secondary amine (e.g. methyl (3,5-dibromo-4-{3-(isopropylamino)-4-[(3-methylbutanoyl)amino]phenoxy}phenoxy)acetate), was cyclized without further purification.

The ethyl alkylation of the aniline (e.g. methyl (3,5-dibromo-4-{3-(ethylamino)₄-[(3-methylbutanoyl)amino]phenoxy}phenoxy)acetate) was obtained as a secondary product in this reaction and it was cyclized using the same procedure

A solution of the appropriate secondary amine derivative (e.g. methyl (3,5-dibromo-4-{3-(isopropylamino)-4-[(3-methylbutanoyl)amino]phenoxy}phenoxy)acetate) in acetic acid (50 mL/mmol) was stirred for 17 h at 80° C. After evaporation of the acetic acid, the residue was dissolved in tetrahydrofuran and lithium hydroxide (IM) was added. After stirring for 17 h at 20° C., LCMS analysis revealed that the starting material was consumed. After acidification with hydrochloric acid (2M) the product was extracted into ethyl acetate. The combined organic layers were washed with brine and dried over sodium sulphate. After filtration, the residue was purified by semi-preparative-HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) to give the expected acid (e.g. {3,5-dibromo-4-[(2-isobutyl-1-isopropyl-1H-benzimidazol-6-yl)oxy]phenoxy}acetic acid).

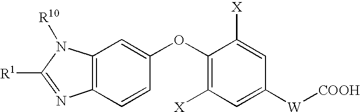

| | | | | | Yield | MW | M + 1 |
|---|---|---|---|---|---|---|---|
| Example | R¹ | R¹⁰ | X | W | (%) | (calc) | (found) |
| 39 | Me | i-Pr | Br | (CH₂)₂ | 3 | 496.2 | 497.6 |
| 40 | Me | Et | Br | (CH₂)₂ | 1 | 482.2 | 483.2 |
| 41 | i-Bu | i-Pr | Br | O—CH₂ | 6 | 540.3 | 541.4 |
| 42 | i-Bu | Et | Br | O—CH₂ | 3 | 526.2 | 527.3 |
| 43 | p-Cl-Ph | Et | Br | (CH₂)₃ | 5 | 592.7 | 591.5 |
| | | | | | | | (M − 1) |
| 44 | i-Bu | i-Pr | Br | CH₂—CHF | 11 | 556.3 | 557.1 |
| 45 | Cyclo- | i-Pr | Br | CH₂—CHF | 8 | 582.3 | 583.1 |
| | propyl- | | | | | | |
| | methyl | | | | | | |
| 46 | 2-Methyl- | Et | Br | CH₂—CHF | 19 | 560.3 | 561 |
| | thio- | | | | | | |
| | ethyl | | | | | | |
| 47 | i-Bu | Et | Br | CH₂—CHF | 11 | 578.3 | 579.5 |

### Example 47

### 3-[3,5-dibromo-4-({1-ethyl-2-[(methylsulfonyl)methyl]-1H-benzimidazol-6yl}oxy)phenyl]-2-fluoropropanoic acid

To a solution of methyl 3-[4-(4-Amino-3-nitro-phenoxy)-3,5-dibromo-phenyl]-2-fluoro-propanoate (55 mg, 0.1 mmol) in dichloromethane (5 mL), pyridine (22 μL, 2.5 equiv) and chloroacetyl chloride (17 μL, 1.1 equiv) were added. The reaction was stirred over night at ambient temperature. To the reaction mixture, dichloromethane and hydrochloric acid (2M) were added and the phases separated with a phase separator. After evaporation the crude product was filtrated on a silica SPE column (heptane/ethyl acetate 7:3) giving 50 mg of methyl 3-{3,5-dibromo-4-[4-(2-chloro-acetylamino)-3-nitro-phenoxy]-phenyl}-2-fluoro-propanoate as a yellow solid which was used without further purification. Yield 85%.

To a solution of methyl 3-{3,5-dibromo-4-[4-(2-chloro-acetylamino)-3-nitro-phenoxy]-phenyl}-2-fluoro-propanoate (50 mg, 0.09 mmol) in dimethylformamide (2 mL), potassium carbonate (18 mg, 1.3 equiv) was added, followed by addition of methane sulphinic acid sodium salt (10 mg, 1. I equiv). The reaction was left for 72 h at ambient temperature. The aqueous mixture was acidified to pH close to I causing precipitation. Dichloromethane was added (5×50 mL) and the combined organic phases were washed with brine. The organic layer was dried over sodium sulphate, the solids were filtered, and the filtrate was concentrated under vacuum. The resulting residue was dissolved and evaporated on silica. Filtration through silica (0%-10% methanol in dichloromethane) gave 45 mg of methyl 3-{3,5-dibromo-4-[4-(2-methanesulfonyl-acetylamino)-3-nitro-phenoxy]-phenyl}-2-fluoro-propanoate.

To a stirred solution of methyl 3-{3,5-dibromo-4-[4-(2-methanesulfonyl-acetylamino)-3-nitro-phenoxy]-phenyl}-2-fluoro-propanoate) (45 mg, 0.074 mmol) in a 1:9 water/acetic acid (3.5 mL) was added iron powder (20 mg, 5 equiv). After stirring for 5 h at 20° C., LCMS analysis revealed that the starting material was consumed. The solution was diluted with ethyl acetate (20 mL) and water (20 mL). Hydrochloric acid (2M, 1 mL) was added, the phases were separation and the water phase was extracted with ethyl acetate (2×50 mL). The combined ethyl acetate layers were washed with brine, dried over sodium sulphate and concentrated under vacuum to yield 35 mg of methyl 3-{4-[3-amino-4-(2-methanesulfonyl-acetylamino)-phenoxy]-3,5-di bromo-phenyl}-2-fluoro-propanoate as a slightly brown solid that was used without further purification. Yield 82%. Sodium cyanoborohydride (11 mg, 3 equiv) was added to the solution of methyl 3-{4-[3-amino-4-(2-methanesulfonyl-acetylamino)-phenoxy]-3,5-dibromo-phenyl}-2-fluoro-propanoate (35 mg, 0.06 mmol) and acetaldehyde (4 μL, 1.2 equiv) in methanol:tetrahydrofuran (2:1, 1.5 mL). The mixture was stirred at room temperature for 17 h. LCMS revealed that the reaction gave mainly product but that starting material remained and a by-product was formed. Mass indicated that this was the dialkylated. In order to avoid more dialkylated by-product the reaction was quenched with saturated ammonium chloride and extracted with ethyl acetate (3×50 mL). Washed with brine and evaporated. The crude was used without further purification.

The crude mixture containing methyl 3-{3,5-dibromo-4-[3-ethylamino-4-(2-methanesulfonyl-acetylamino)phenoxy]-phenyl}-2-fluoro-propanoate (20 mg) was dissolved in acetic acid and heated at 70° C. for 7 h. The reaction was allowed to reach room temperature and the acetic acid was evaporated. The crude oil was dissolved in tetrahydrofuran and lithium hydroxide (IM) was added and the mixture was stirred over night at ambient temperature. The reaction mixture was acidified with hydrochloric acid (2 M) and extracted with ethyl acetate. The crude was purified by semi-preparative-HPLC (Zorbax CombiHT (SB-C8 50×21.2 mm, 5μ) Mobile Phase: Solvent A. Water with 0.5% formic acid; Solvent B: acetonitrile. Gradient: 2 min 80% of A then over 8 min to 5% of A) to give 6.5 mg of 3-[3,5-dibromo-4-({1-ethyl-2-[(methylsulfonyl)methyl]-1H-benzimidazol-6-yl}oxy)phenyl]-2-fluoropropanoic acid as a white solid. Overall yield from 3-[4-(4-Amino-3-nitro-phenoxy)-3,5-dibromo-phenyl]-2-fluoro-propionic acid methyl ester, 10.8%

LC-MS (ESI) M+1_{found}=579.5 (MW_{calc}=578.3)

### Preparation of Benzoxazoles.

### Example 48

### [3,5-dichloro-4-(2-isobutyl-1,3-benzoxazol-6-yl)oxy phenyl]acetic acid

A mixture of methyl[4-(4-amino-3 bromo-phenoxy)-3,5-dichlorophenyl]acetate (80 mg, 0.20 mmol), 3-methyl-butyryl chloride (40 μL, 0.40 mmol) and triethylamine (82 μL, 0.60 mmol) in dichloromethane (2 mL) was stirred at room temperature for 2 h. The reaction mixture was partitioned between hydrochloric acid (1 M) and dichloromethane. The organic layer was separated, dried using a phase separator and concentrated in vacuo. The residual oil was loaded on a silica flash gel chromatography column and purified (diethylether/dichloromethane 0:10 to 5:5) to afford methyl {4-[3-bromo-4-(3-methyl-butyrylamino)-phenoxy]-3,5-dichlorophenyl}acetate (95 mg, 98% yield).

A mixture of methyl {4-[3-bromo-4-(3-methyl-butyrylamino)phenoxy]-3,5-dichlorophenyl}acetate (25 mg, 0.051 mmol), isopropylamine (33 mg, 0.056 mmol), potassium carbonate (14 mg, 0.10 mmol), copper iodide (5 mg, 0.025 mmol) and a L-proline (9 mg, 0.008 mmol) in dimethylsulphoxide (0.5 mL) was heated at 60° C. for 12 h. The cooled mixture was partitioned between water and ethyl acetate. The organic layer was separated, and the aqueous layer was extracted with ethyl acetate. The combined organic layers were dried over magnesium sulphate and concentrated in vacuo. The residual oil was dissolved in tetrahydrofuran (2 mL) and potassium hydroxide (2 M, 6 mL) was added. The mixture was stirred for 1 h at room temperature. The final product was purified by semi-preparative-HPLC (Zorbax CombiHT (SB-C8 50×21.2 mm, 5μ) Mobile Phase Solvent A. Water with 0.5% formic acid; Solvent B: acetonitrile. Gradient: 2 min 80% of A then over 8 min to 5% of A) to give [3,5-dichloro-4-{(2-isobutyl-1,3-benzoxazol-6-yl)oxy}phenyl]acetic acid (2 mg, 10% yield).

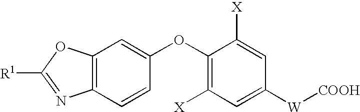

| | | | | Yield | MW | M |
|---|---|---|---|---|---|---|
| Example | R¹ | X | W | (%) | (calc) | (found) |
| 48 | i-Bu | Cl | CH₂ | 10 | 394.2 | 394.2 |

### Preparation of Quinazolines.

### Example 49

### 3-{3,5-dibromo-4-[(quinazolin-6-yl)oxy]phenyl}propanoic acid

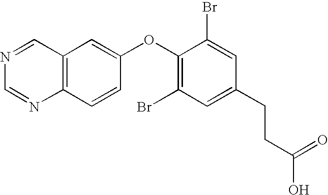

A mixture of methyl 3-[4-(4-aminophenoxy)-3,5-dibromophenyl]propanoate (52 mg, 0.12 mmol), ethyl chloroformate (23 μL, 0.24 mmol) and triethylamine (34 mL, 0.24 mmol) in tetrahydrofuran (1 mL) was stirred at room temperature for 2 h. Water and ethyl acetate were added and the organic layer was separated. The aqueous layer was extracted with ethyl acetate (3×5 mL). The combined organic layers were dried over magnesium sulphate and concentrated in vacuo. The residual oil was loaded on a silica flash gel chromatography column and purified (diethylether/dichloromethane 0:10 to 5:5) to afford methyl 3-[4-(4-ethoxycarbonylamino-phenoxy)-3,5-dibromophenyl]propanoate (40 mg, 66% yield).

A mixture of methyl 3-[4-(4-ethoxycarbonylamino-phenoxy)-3,5-dibromophenyl]propanoate (40 mg, 0.080 mmol) and hexamethylenetetramine (74 mg, 0.53 mmol) in trifluoroacetic acid (100 μL) was heated at 90° C. for 12 h. After cooling, the mixture was diluted with sat. sodium bicarbonate and extracted with ethyl acetate (3×5 mL). The organic layer were dried over magnesium sulphate and concentrated in vacuo.

The aqueous ethanolic solution of the obtained residue (water/ethanol:1/1) was treated with potassium hydroxide (4 mL, 10%) and the mixture was refluxed for 1 h. After cooling, potassium hexacyanoferrate (III) (0.13 g; 0.40 mmol) was added and the mixture was refluxed for 12 h. After cooling, the reaction mixture was partitioned between hydrochloric acid (IM) and ethyl acetate and the organic layer was separated. The aqueous layer was extracted with ethyl acetate (3×5 mL). The combined organic layers were dried over magnesium sulphate and concentrated in vacuo. The residual oil was purified by semi-preparative-HPLC (Zorbax CombiHT (SB-C8 50×21.2 mm, 5μ) Mobile Phase Solvent A. Water with 0.5% formic acid; Solvent B: acetonitrile. Gradient: 2 min 80% of A then over 8 min to 5% of A) to afford of 3-{3,5-dibromo-4-[(quinazolin-6-yl)oxy]phenyl}propanoic acid (1.47 mg, 4% yield).

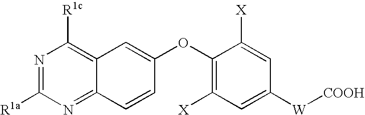

| | | | | | Yield | MW | M − 1 |
|---|---|---|---|---|---|---|---|
| Example | R^{1a} | R^{1c} | X | W | (%) | (calc) | (found) |
| 49 | H | H | Br | (CH₂) | 4 | 452.1 | 451.0 |

### Preparation of Indazoles.

### Intermediate 6

### 3,5-dibromo-4-(2-phenyl-2H-indazol-5-yloxy-phenylamine

To phenylhydrazine (1.080 g, 10 mmol) stirred at 0° C., LiRMDS (10 ml, Lithium bis (trimethylsilyl)amide, 1M in THF) was added slowly via a syringe. The reaction mixture was stirred at 0° C. for 15 min. Then, 2-bromo-5-methoxybenzyl bromide (840 mg, 3 mmol) was added in portions. The mixture was stirred at room temperature overnight. The reaction was quenched by addition of water (20 mL). The mixture was extracted with dichloromethane (3×30 mL). The combined organic layers washed with brine (2×30 mL) and dried over magnesium sulphate. Evaporation of the solvent yielded a crude product (N-(2-bromo-5-methyoxy-phenyl)-N-phenyl-hydrazine) as yellow oil (1.105 g), which was used without further purification.

To a stirring solution of N-(2-bromo-5-methyoxy-phenyl)-N-phenyl-hydrazine (1.105 g, 3.1 mmol) in dry toluene (12 mL) at room temperature, palladium acetate (37 mg, 0.16 mmol) and of dppf (137 mg, 0.24 mmol, 1,1′-bis(diphenylphosphino)ferrocene) were added. The mixture was stirred for 5 min. and sodium tertbutoxide (396 mg, 4 mmol) was added. The reaction mixture was stirred at 90° C. for 48 h. The reaction mixture was diluted with ethyl acetate (100 mL). The organic phase was washed with brine (3×50 mL), dried over magnesium sulphate and the solvents evaporated under vacuum. The resulting residue was purified by flash chromatography (ethyl acetate/heptane 20:80) to yield 210 mg of 5-methoxy-2-phenyl-2H-indazole.

To a 0° C. solution of 5-methoxy-2-phenyl-2H-indazole (160 mg, 0.7 mmol) in dichloromethane (5 mL) BF₃ SMe₂ complex (1.2 mL, 9 mmol) was added. The mixture was stirred at room temperature overnight. Ethyl acetate (30 mL) was added to quench the reaction. The organic layer was washed with brine (3×20 mL) and dried over magnesium sulphate. The solvent was evaporated under vacuum and the residue was purified by flash chromatography (ethyl acetate/heptane 30:70) to yield of 2-phenyl-2H-indazol-5-ol (131 mg).

2-phenyl-2H-indazol-5-ol (91 mg, 0.43 mmol) and 1,3-dibromo-2-iodo-5-nitro-benzene (175 mg, 0.43 mmol) were dissolved in dimethylformamide (5 mL), and potassium carbonate (138 mg, 1.29 mmol) was added. The mixture was stirred at 70° C. overnight. Ethyl acetate (25 mL) was added. The organic layer was washed with ammonium chloride (sat. aq, 10 mL) and brine (2×10 mL), dried over magnesium sulphate and the solvents evaporated under vacuum. The residue was purified by flash chromatography (ethyl acetate/heptane 20:80). 42 mg of 5-(2,6-dibromo-4-nitro-phenoxy)-2-phenyl-2H-indazole were obtained.

A solution of 5-(2,6-dibromo-4-nitro-phenoxy)-2-phenyl-2H-indazole (42 mg, 0.09 mmol) in methanol (17 mL) was treated with tin(II) chloride (113 mg, 0.5 mmol) heated to reflux overnight. The reaction mixture was cooled to room temperature and neutralized with sodium bicarbonate (sat. aq.). Methanol was removed under vacuum and the water phase was extracted with ethyl acetate (3×15 mL). The combined organic layers were washed with brine (3×10 mL) and dried over magnesium sulphate. The evaporation of solvent yielded a crude 3,5-dibromo-4-(2-phenyl-2H-indazol-5-yloxy-phenylamine (37 mg. 5% total yield).

### Example 50

### 3-({3,5dibromo-4-[(2-phenyl-2H-indazol-5-yl)oxy]phenyl}amino)-3-oxopropanoic acid

To a 0° C. dichloromethane (4 mL) solution of 3,5-dibromo-4-(2-phenyl-2H-indazol-5-yloxy-phenylamine (37 mg, 0.08 mmol) were added triethylamine (10 mg, 0.10 mmol) and chlorocarbonyl acetic acid methyl ester (14 mg, 0.10 mmol). The mixture was stirred at 0° C. for 3 h. The reaction was quenched with ammonium chloride (1 mL, sat. aq.) and ethyl acetate was added (25 mL). The organic layer was washed with brine (3×10 mL), dried over magnesium sulphate and concentrated under vacuum. The residue was purified by flash chromatography (ethyl acetate/heptane 40:60) to afford 41 mg of methyl 3-({3,5-dibromo-4-[(2-phenyl-2H-indazol-5-yl)oxy]phenyl}amino)-3-oxopropanoate.

Methyl 3-({3,5-dibromo-4-[(2-phenyl-2H-indazol-5-yl)oxy]phenyl}amino)3-oxopropanoate (41 mg, 0.07 mmol) was mixed with 1,4-dioxane (1 mL) and sodium hydroxide (1M, 6 mL) and stirred at room temperature overnight. The reaction mixture was neutralized with hydrochloric acid (IM) to pH 5-6 and extracted with ethyl acetate (3×20 mL). The combined organic layers were washed with brine (3×15 mL) and the solvent evaporated under vacuum. The resulting residue was purified using semi-preparative-HPLC (Zorbax CombiHT(SB-C8 50×21.2 mm, 51) Mobile Phase: Solvent A. Water with 0.5% formic acid; Solvent B: acetonitrile. Gradient: 2 min 80% of A then over 8 min to 5% of A) to afford 17 mg of 3-({3,5-dibromo-4-[(2-phenyl-2H-indazol-5-yl)oxy]phenyl}amino)-3-oxopropanoic acid. Yield: 40%

### Example 51

### N-{3,5-dibromo-4-[(2-phenyl-2H-indazol-5-yl)oxy]phenyl}glycine

A mixture of 3,5-dibromo-4-(2-phenyl-2H-indazol-5-yloxy-phenylamine (190 mg, 0.4 mmol), ethyl bromoacetate (67 mg, 0.4 mmol), sodium iodide (30 mg, 0.2 mmol) and potassium carbonate (170 mg, 1.2 mmol) in dimethylformamide (3 mL) was placed in a vial. The vial was flushed with nitrogen and sealed. The mixture was heated at 100° C. for 4 h and then cooled to room temperature.

The reaction mixture was extracted with ethyl acetate (3×15 mL). The organic phase was washed once with brine and dried over sodium sulphate. Concentration of the organic phase gave ethyl N-{3,5-dibromo-4-[(2-phenyl-2H-indazol-5-yl)oxy]phenyl}glycinate as a yellow solid (252 mg) which was used in the next step without further purification.

To a room temperature solution of the crude ethyl N-{3,5-dibromo-4-[(2-phenyl-2H-indazol-5-yl)oxy]phenyl}glycinate in tetrahydrofuran (6 mL) and methanol (3 mL), sodium hydroxide (7 mL, 2N) was added. After 3 h, the reaction mixture was concentrated and the residue was dissolved in a mixture ethyl acetate/1N hydrochloric acid. The phases were separated and the acidic phase was extracted with ethyl acetate (2×5 mL). The organic layers were combined, washed with brine, dried over anhydrous sodium sulphate and evaporated. The residue was purified on silica gel column (dichloromethane/methanol, 10:1) to give 67 mg of N-{3,5-dibromo-4-[(2-phenyl-2H-indazol-5-yl)oxy]phenyl}glycine as a white solid. Yield for two steps: 33%.

### Example 52

### {3,5-dibromo-4-[(2-phenyl-2H-indazol-5-yl)oxy]phenyl}acetic acid

A mixture of 3,5-dibromo-4-(2-phenyl-2H-indazol-5-yloxy-phenylamine (480 mg, 1 mmol), ethanol (20 mL), hydrochloric acid (37%, 20 mL) and water (20 ml) was placed at 0° C. A suspension of sodium nitrite (128 mg, 1.5 mmol) in water (2 mL) was added and the resulting reaction mixture was stirred for 15 min at 0° C. A solution of potassium iodide (498 mg, 3 mmol) in water (2 mL) was added and the mixture was allowed to stir for another hour. The reaction mixture was concentrated and the residue was extracted with ethyl acetate (3×10 mL), washed with brine and dried over sodium sulphate. The residue obtained after evaporation of the solvents was purified by flash chromatography (ethyl acetate/petroleum ether, 20:1) to give 5-(2,6-dibromo-4-iodo-phenoxy)-2-phenyl-2H-indazole (410 mg, yellow solid) (72% yield).

To a solution of dimethyl malonate (200 mg, 1.5 mmol) in dimethylformamide (10 mL) at 0° C., sodium hydride (52 mg, 1.5 mmol) and 5-(2,6-Dibromo-4-iodo-phenoxy)-2-phenyl-2H-indazole (300 mg, 0.5 mmol) were added. The suspension was stirred for 1 h at 0° C. Then, copper chloride (200 mg, 2 mmol) was added and the solvent was warmed gently to 100° C. overnight. The reaction mixture was quenched with hydrochloric acid (IN) and poured into a separatory funnel containing brine/ethyl acetate. The phases were separated and the acidic phase was extracted with ethyl acetate

(2×10 mL). The combined organic layers were washed with brine, dried over anhydrous sodium sulphate and concentrated under vacuum. The residue was purified by flash chromatography (petroleum ether/ethyl acetate, 8:1) to give 2-[3,5-Dibromo-4-(2-phenyl-2H-indazol-5-yloxy)-phenyl]-malonic acid dimethyl ester (68 mg, 24% yield)

A vial containing a solution of 2-[3,5-Dibromo-4-(2-phenyl-2H-indazol-5-yloxy)-phenyl]-malonic acid dimethyl ester (68 mg, 0.12 mmol) in dimethylsulphoxide (4 mL) was flushed with nitrogen and sealed. The mixture was heated at 150° C. for 3 h and then cooled to room temperature. The reaction mixture was extracted with ethyl acetate (3×5 mL). The organic phase was washed with brine, dried over sodium sulphate and concentrated under vacuum. The resulting residue was purified by flash chromatography (petroleum ether/ethyl acetate, 8:1) to give [3,5-Dibromo-4-(2-phenyl-2H-indazol-5-yloxy)-phenyl]-acetic acid methyl ester (53 mg, 85% yield). To a tetrahydrofuran (4 mL) solution of methyl {3,5-dibromo-4-(2-phenyl-2H-indazol-5-yl)oxy]phenyl}acetate (53 mg, 0.1 mmol) in at room temperature, methanol (2 mL) and sodium hydroxide (2N, 2 mL) were added. After 3 h, the reaction mixture was concentrated under vacuum and the residue was dissolved in a mixture ethyl acetate/hydrochloric acid (IN). The phases were separated and the acidic phase was extracted with ethyl acetate (2×5 mL). The combined organic layers were washed with brine, dried over anhydrous sodium sulphate and the solvents evaporated under vacuum. The obtained residue was purified on silica gel column chromatography (dichloromethane/methanol, 10:1) to give {3,5-dibromo-4-[(2-phenyl-2H-indazol-5-yl)oxy]phenyl}acetic acid as a white solid (41 mg, 80% yield).

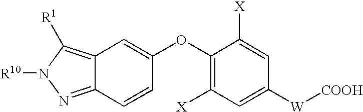

| | | | | | Yield | MW | M |
|---|---|---|---|---|---|---|---|
| Example | R¹ | R¹⁰ | X | W | (%) | (calc) | (found) |
| 50 | H | Ph | Br | NHCOCH₂ | 40 | 545.2 | 544.4 |
| | | | | | | | (M − 1) |
| 51 | H | Ph | Br | NH—CH₂ | 32 | 517.1 | 517.9 |
| 52 | H | Ph | Br | CH₂ | 12 | 502.2 | 502.6 |
| 53 | MeNHCO | Me | Br | NHCOCH₂ | 9 | 540.1 | 538 |
| | | | | | | | (M − 2) |

### Example 53

### 3-{[3,5-dibromo-4-({2-methyl-3-[(methylamino)carbonyl]-2H-indazol-5-yl}oxy)phenyl]amino}-3-oxopropanoic acid

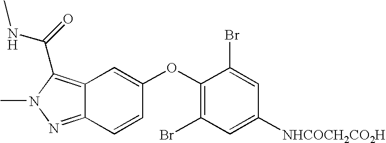

2-methyl-4-methoxyphenylamine (27.4 g, 200 mmol) was added to a solution of tetrafluoroboric acid (HBF₄, 50% aqueous solution, 100 mL). The solution was stirred at room temperature for about min, then cooled to 0˜5° C. A solution of sodium nitrite (13.9 g, 200 mmol) in water (20 mL) was dropped in. The mixture was warmed to room temperature and stirred 1 h. The reaction mixture was filtrated and the crude product was washed with diethyl ether (3×100 mL) and dried in air to provided 49.7 g of 2-methyl-4-methoxyphenyldiazonium tetrafluoroborate.

2-methyl-4-methoxyphenyldiazonium tetrafluoroborate (49.7 g, 211 mmol), 18-crown-6 (2.79 g, 10.6 mmol), potassium acetate (43.4 g, 422 mmol) were added to chloroform (300 mL). The reaction mixture was stirred at room temperature for 2 h. The solution was washed with brine (3×30 mL), dried over sodium sulphate, and the solvents evaporated under vacuum. The residue was purified by flash chromatography (ethyl acetate/petroleum ether 2:8 to 4:6) to provide 5-methoxy-1H-indazole (10.2 g). LC-MS (ESI) M+1_{found}=149 (MW_{calc}=148.1)

To a stirred mixture of 5-methoxy-1H-indazole (9.5 g, 64.6 mmol) in ethyl acetate (200 mL), was added trimethyloxonium tetrafluoroborate (19.1 g, 129 mmol). The mixture was stirred at room temperature for 2 h. The reaction mixture was washed with saturated NaHCO₃ solution (100 mL). The organic layer was separated and the aqueous layer was extracted with ethyl acetate (2×100 mL). The combined organic layers were dried over anhydrous sodium sulphate, filtered and the solvents evaporated. Purification of the residue by flash chromatography (ethyl acetate/petroleum ether 2:3) gave 5-methoxy-2-methyl-2H-indazole (8.6 g). LC-MS (ESI) M+1_{found}=163 (MW_{calc}=162.1).

To a mixture of 5-methoxy-2-methyl-2H-indazole (8.2 g, 50.6 mmol) in acetic acid (100 mL) was added N-bromosuccinimide (9.01 g, 50.6 mmol). The mixture was stirred at room temperature for 4 h. The reaction was quenched with ethyl acetate (200 mL) and washed with saturated NaHCO₃ aqueous solution until stopped bubbling. The organic layer was separated and washed with brine, then dried over anhydrous sodium sulphate, filtered and concentrated under vacuum. Purification of the residue by flash chromatography (ethyl acetate/petroleum ether 1:9) gave 3-bromo-5-methoxy-2-methyl-2H-indazole (8.23 g). LC-MS (ESI) M_{found}=241 (MW_{calc}=241.1)
3-Bromo-5-methoxy-2-methyl-2H-indazole (7.9 g, 32.7 mmol) was dissolved in dimethylacetamide (200 mL), and the following reagents were added: Pd₂(dba)₃ (1.2 g, 1.3 mmol, 4 mol %), Dppf (1.4 g, 2.6 mmol, 8 mol %), Zn powder (513 mg, 7.8 mmol, 24 mol %) and Zn(CN)₂ (4.6 g, 39.2 mmol). The mixture was stirred at 170° C. for 6 h. The reaction mixture was quenched with water (400 mL) and extracted with ethyl acetate (3×200 mL). The organic extracts were dried over sodium sulphate and concentrated in vacuum. The crude product was purified by flash chromatography (ethyl acetate/petroleum ether 2:8) to give 5-methoxy-2-methyl-2H-indazole-3-carbonitrile as a white solid (5.9 g).

5-Methoxy-2-methyl-2H-indazole-3-carbonitrile (4.67 g, 25 mmol) was dissolved in methanol (60 mL) and an aqueous solution of sodium hydroxide (10%, 60 mL) was added. The reaction mixture was refluxed for 4 h. Methanol was evaporated in vacuum. The residue was acidified to pH=4-5 and extracted with ethyl acetate (3×100 mL). The combined organic layers were washed with brine, dried and evaporated to provide 5-methoxy-2-methyl-2H-indazole-3-carboxylic acid (4.3 g) as a white powder.

To a dichloromethane (400 mL) solution of 5-methoxy-2-methyl-2H-indazole-3-carboxylic acid (4.3 g, 20.6 mmol) were added methylamine (hydrochloride salt, 2.8 g, 41.3 mmol), 1-hydroxybenzotriazole hydrate (HOBt) (5.6 g, 41.3 mmol), 3-ethyl-1-[3-(dimethylamino)propyl]carbodiimide hydrochloride (EDCI) (11.9 g, 62 mmol) and triethylamine (17 mL, 124 mmol). The reaction mixture was stirred at room temperature for 3 h and then quenched with water (200 mL). The organic layer was separated and the aqueous layer was extracted with dichloromethane (2×100 mL). The combined organic layers were washed with diluted hydrochloric acid and brine, dried and evaporated to provide 5-methoxy-2-methyl-2H-indazole-3-carboxylic acid methylamide (3.03 g). LC-MS (ESI) M_{found}=219 (MW_{calc}=219.2) To a solution of 5-methoxy-2-methyl-2H-indazole-3-carboxylic acid methylamide (2.9 g, 13.1 mmol) in dry dichloromethane (150 mL), Boron trifluoride-methyl sulfide complex (1M, 35 mL) was dropped in at 0° C. and the reaction mixture was warmed to room temperature and stirred overnight. The reaction was quenched with water, the water phase was extracted with dichloromethane (3×50 mL), and the combined organic phases were washed with brine, dried over sodium sulphate, and concentrated under vacuum to provide 5-hydroxy-2-methyl-2H-indazole-3-carboxylic acid methylamide (2.7 g).

Yield from 2-methyl-4-methoxyphenylamine: 10%

To a solution of 5-hydroxy-2-methyl-2H-indazole-3-carboxylic acid methylamide (820 mg, 4 mmol) in dimethylformamide (20 mL), 1,3-dibromo-2-iodo-5-nitro-benzene (1.6 g, 4 mmol) and potassium carbonate (1.660 g, 12 mmol) were added. The reaction mixture was stirred at 70° C. for 2 h and then quenched with water (100 mL). The water phase was extracted with ethyl acetate (3×20 mL), the combined organic phases were washed with brine, dried over sodium sulphate and concentrated under vacuum to provided 1.18 g of the wanted compound 5-(2,6-dibromo-4-nitro-phenoxy)-2-methyl-2H-indazole-3-carboxylic acid methylamide.

To a ethanol (100 mL) solution of 5-(2,6-dibromo-4-nitro-phenoxy)-2-methyl-2H-indazole-3-carboxylic acid methylamide (110.18 g, 2.4 mmol), tin(II) chloride dehydrate (2.7 g, 12.2 mmol) was added. The reaction mixture was stirred at 60° C. overnight, then cooled to room temperature and the solvent evaporated. The reaction mixture was poured into aqueous sodium hydroxide solution (10%, 150 mL) and extracted with ethyl acetate (3×100 mL). The combined organic phases were washed with brine (50 mL), dried over sodium sulphate and the solvents evaporated under vacuum. Purification by flash chromatography (ethyl acetate) provided 327 mg of 5-(4-Amino-2,6-dibromo-phenoxy)-2-methyl-2H-indazole-3-carboxylic acid methylamide. LC-MS (ESI) M-2_{found}=452 (MW_{calc}=454.1)

To a dichloromethane (10 mL) solution of 5-(4-Amino-2,6-dibromo-phenoxy)-2-methyl-2H-indazole-3-carboxylic acid methylamide (112 mg, 0.24 mmol), pyridine (40 ul, 0.49 mmol) and methyl 3-chloro-3-oxopropionate (26 ul, 0.24 mmol) were added. After 0.5 h stirring at room temperature, dichloromethane was evaporated and ethyl acetate added. The mixture was washed with water followed by saturated brine, dried over sodium sulphate and then concentrate under vacuum. The residue was purified by flash chromatography (ethyl acetate) to afford 112 mg of compound methyl 3-{[3,5-dibromo-4-({2-methyl-3-[(methylamino)carbonyl]-2H-indazol-5-yl}oxy)phenyl]amino}-3-oxopropanoate.

To a methanol/tetrahydrofuran (10 mL/10 mL) solution of methyl 3-{[3,5-dibromo-4-({2-methyl-3-[(methylamino)carbonyl]-2H-indazol-5-yl}oxy)phenyl]amino}-3-oxopropanoate (112 mg, 0.2 mmol, aqueous sodium hydroxide solution (1N, 1 mL, 1 mmol) was added. The reaction mixture was stirred 3 h at room temperature. Methanol and tetrahydrofuran were evaporated under vacuum and water was added. The mixture was acidified to pH67 with hydrochloric acid (2N) and extracted with ethyl acetate, dried and evaporated under vacuum. The crude was re-crystallized with ethyl acetate/hexane to provide 3-([3,5-dibromo-4-({2-methyl-3-[(methylamino)carbonyl]-2H-indazol-5-yl}oxy)phenyl]amino)-3-oxopropanoic acid (63 mg).

LC-MS (ESI) M-2_{found}=538 (MW_{calc}=540.1)

Yield from 5-hydroxy-2-methyl-2H-indazole-3-carboxylic acid methylamide: 9%4

### Examples 54 and 55

### Chiral HPLC Separation

Example 44 (3-{3,5-dibromo-4-[(2-isobutyl-1-isopropyl-1H-benzimidazol-6-yl)oxy]phenyl}-2-fluoropropanoic acid) is a racemic mixture. The single enantiomers were separated by chiral HPLC, providing examples 54 and 55.

### HPLC Analyses

ReproSil Chiral-NR (4.6 mm id×250 mm, 8 μm (Dr. Maisch GmbH, Ammerbuch, Germany)) was used for HPLC analyses. Analyses were carried out using n-heptane: 2-propanol:trifluoroacetic acid (85:10:0.1%) as a mobile phase at a flow rate of 0.8 mL/min and room temperature. Detection was carried out at UV 284 nm. Under these conditions, the retention times were as follows:

Example 54: F1=67 min.

Example 55: F2=77 min

### HPLC Preparative Separation

A column ReproSil Chiral-NR (20 mm id×250 mm, 8 μm (Dr. Maisch GmbH, Ammerbuch, Germany)) with a pre-column ReproSil Chiral-NR (30 mm id×20 mm, 8 μm (Dr. Maisch GmbH, Ammerbuch, Germany)) were used for HPLC preparative separation. The separations were carried out using n-heptane: 2-propanol:trifluoroacetic acid (85:10:0.1%) as a mobile phase at a flow rate of 10.0 mL/min and room temperature. Detection was carried out at UV 284 nm. Under these conditions, the retention times were as follows:

Example 54: F1=118 min.

Example 55: F2=132 min

### CONSULTED LITERATURE

Med. Chem.; 45; 21; 2002; 4647.
J. Chem. Soc. Perkin Trans. 1988, 3229)
Russ. Chem. Bull., Int. ed.; 51; 1; 2002; 144.
J. Chem. Soc. Perkin Trans. 1; 1993; 2747.
J. Am. Chem. Soc. v.125, 2003, 10243
Organic Letters 2006, 255.
Organic Letters 2003, 2453.
Organic Letters 2003, 4257.
J. Org. Chem. 1999, 8588.
J. Org. Chem. 2003, 68, 4093
J. Med. Chem. 2005, 48, 1132
   TR Competition Binding Assay with Filter Separation

Compounds are tested for their ability to compete with the tracer ¹²⁵I-T₃ for binding to fill length hTRα and hTRβ. Receptor extracts and tracer are diluted in assay buffer (17 mM K₂HPO₄, 3 mM KH₂PO₄, 400 mM KCl, 1 mM MgCl₂, 0.5 mM EDTA and 8.7% glycerol). ¹²⁵I-T₃ is diluted to a final concentration of 0.2 nM and receptor is diluted to reach a final count in Trilux Microbeta of approximately 10000 ccpm. Compounds are typically serially diluted in DMSO from DMSO stock solutions of 10 mM. To 96 well microtiter plates are 100 μl tracer, 4 μl test compound dilution series and 100 μl receptor dilution added. The assay plates are incubated at +4° C. over night (app. 16 hrs incubation). Receptor bound and free tracer are separated over a glass fiber filter (FILTERMAT B, PerkinElmer)) on a Tomtec Cellharvester with 18 mM K₂HPO₄, 2 mM KH₂PO₄, 0.5 mM EDTA wash buffer. The filters are dried at 60° C. for 1 hour and then merged with a scintillant wax (MELTILEX, PerkinElmer) on a Wallac Microsealer before measuring in a Trilux Microbeta. ICSOs, the concentration test compound needed to decrease tracer binding by 50 percent, are generated via analysis of data in XLfit version 2.0 or later with a four parameter logistic model. The compounds of the examples were tested in this assay and were found to have an IC₅₀ at the thyroid receptor-beta receptor in the range of from 0.03 nM to 6 μM. Preferred compounds of the invention were found to have an IC₅₀ at the thyroid receptor-beta receptor in the range of from 0.03 nM to 700 nM. Particularly preferred compounds of the invention were found to have an IC₅₀ at the thyroid receptor-beta receptor in the range of from 0.03 nM to 100 nM.

### Vector Constructs, Generation of Reporter Cell Lines (TRAF), and Assay Procedure.

The cDNAs encoding the full length human ThRα1 and ThRβ1 were cloned in the mammalian expression vector pMT-hGH. The pDR4-ALP reporter vector contains one copy of the direct repeat sequence AGGTCA nnnnAGGTCA, fused upstream of the core promoter sequences of the mouse mammary tumor virus long terminal repeat (MMTV), replacing the glucocorticoid response elements. The DR4-MMTV promoter fragment was then cloned in the 5′ end of the cDNA encoding human placental alkaline phosphatase (ALP), followed in the 3′-end by the polyA-signal sequence of the human growth hormone gene. Chinese hamster ovary (CHO) KI cells (ATCC No. CCL 61) were transfected in two steps, first with the receptor expression vectors pMT-hThRα1 and pMT-ThRβ1, respectively, and the drug resistance vector pSV2-Neo, and in the second step, with the reporter vector pDR4-ALP and the drug resistance vector pKSV-Hyg. Individual drug resistant clones were isolated and selected based on T₃ inducibility. One stable reporter cell clone each of CHO/hThRα1 and CHO/hThRβ1 were chosen for further study in response to various thyroid hormone agonists.

### Assay Procedure:

CHO/hThRα1 and CHO/hThRβ1 were seeded in growth medium (Coon's/F12, 10% L-3,5,3′-triiodothyronine and L-thyroxine depleted FCS, 2 mM L-glutamine) in 96-well plates at 20×10³ cells per well. After 24 hours incubation at 37° C. in humidified chambers, at 5% CO₂, conditioned medium was replaced by induction medium (OptiMEM, 2 mM L-glutamine, 50 Um/ml gentamycin) and cells were exposed to the test compounds at serial dilutions, at final DMSO concentration of 0,5%, or to serial dilution of T₃ (positive control), to assess agonist activity of test compounds. In order to examine antagonistic effect of test compounds, CHO/hThRα1 and CHO/hThRβ1 cells were exposed to serial dilution of the compounds in the presence of 1 nM T₃ (CHO/hThRα1) or 3 nM T₃ (CHO/hThRβ1).

After 48 hours incubation at 37° C. in humidified chambers at 5% CO₂ the level of alkaline phosphatase expressed and secreted into the cell culture medium was analyzed by chemiluminescence on MicroBeta Trilux. The compounds were found to exhibit agonism of at least 12% at the thyroid receptor-beta. Preferred compounds of the invention were found to exhibit agonism of over 40% at the thyroid receptor-beta. Further preferred compounds of the invention were found to exhibit agonism of over 60% at the thyroid receptor-beta.

1. A compound of formula (I) or a pharmaceutically acceptable ester, amide, solvate or salt thereof, including a salt of such an ester or amide, and a solvate of such an ester, amide or salt, 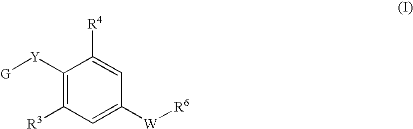
wherein:
G is a group selected from: 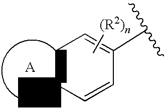
N is a sp² nitrogen with a non-bonded electron pair in an sp² orbital;
The ring A is an aromatic or a non-aromatic five-membered or six-membered ring optionally comprising one or more further heteroatoms independently selected from oxygen, sulfur, sp² nitrogen, and —N(R¹⁰)—, the carbon atoms of ring A optionally being substituted with one or more groups R¹;
Each R¹⁰ is independently selected from —(CH₂)ₚ—S—R^{b}, —(CH₂)ₚ—SO₂—R^{b}, —(CH₂)ₚ—NH—SO₂—R^{b}, —(CH₂)ₚ—SO₂—NH—R^{b}, —(CH₂)ₚ—NH—CO—R^{b}, —(CH₂)ₚ—CO—NH—R^{b}, C₁₋₁₂ alkyl, C₂₋₁₂ alkenyl, C₂₋₁₂ alkynyl, C₃₋₈ cycloalkyl, C₃₋₈ cycloalkyl-C₁₋₃ alkyl, phenyl, benzyl and C₃₋₇heterocyclyl, said alkyl, alkenyl or alkynyl groups or portions of groups optionally being substituted with 1, 2 or 3 groups each independently selected from halogen, hydroxy, N(R^{a})₂, phenyl, C₁₋₄ alkoxy, haloC₁₋₄ alkoxy, dihaloC₁₋₄ alkoxy, and trihaloC₁₋₄ alkoxy; said cycloalkyl, phenyl, benzyl or heterocyclyl groups or portions of groups optionally being substituted with 1, 2 or 3 groups independently selected from halogen, hydroxy, N(R^{a})₂, C₁₋₄ alkyl, C₂₋₄ alkenyl, C₂₋₄ alkynyl, haloC₁₋₄alkyl, dihaloC₁₋₄alkyl, trihaloC₁₋₄alkyl, methoxy, halomethoxy, dihalomethoxy, and trihalomethoxy;
p is 1 or 2;
each R^{a} is independently selected from a hydrogen atom and a C₁₋₄ alkyl group optionally substituted with 1, 2 or 3 groups independently selected from halogen, methoxy, halomethoxy, dihalomethoxy and trihalomethoxy;
each R^{b} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy and trihalomethoxy;
Each R¹ is independently selected from hydrogen, hydroxy, halogen, N(R^{a})₂, —(CH₂)ₘ—S—R^{b}, —(CH₂)ₘ—SO₂—R^{b}, —(CH₂)ₘ—NH—SO₂—R^{b}, —(CH₂)ₘ—SO₂—NH—R^{b}, —(CH₂)ₘ—NH—CO—R^{b}, —(CH₂)ₘ—CO—NH—R^{b}, C₁₋₁₂ alkyl, C₂₋₁₂ alkenyl, C₂₋₁₂ alkynyl, C₃₋₈ cycloalkyl, C₃₋₈cycloalkyl-C₁₋₃ alkyl, phenyl, benzyl and C₃₋₇heterocyclyl, said alkyl, alkenyl or alkynyl groups or portions of groups optionally being substituted with 1, 2 or 3 groups each independently selected from halogen, hydroxy, N(R^{a})₂, phenyl, C₁₋₄ alkoxy, haloC₁₋₄ alkoxy, dihaloC₁₋₄ alkoxy, and trihaloC₁₋₄ alkoxy; said cycloalkyl, phenyl, benzyl or heterocyclyl groups or portions of groups optionally being substituted with 1, 2 or 3 groups independently selected from halogen, hydroxy, N(R^{a})₂, C₁₋₄ alkyl, C₂₋₄ alkenyl, C₂₋₄ alkynyl, haloC₁₋₄alkyl, dihaloC₁₋₄alkyl, trihaloC₁₋₄alkyl, methoxy, halomethoxy, dihalomethoxy, and trihalomethoxy;
m is 0, 1 or 2;
Each R² is independently selected from halogen, mercapto, cyano, C₁₋₄ alkoxy, C₁₋₄ alkyl, C₂₋₄ alkenyl, C₂₋₄ alkynyl and N(R^{a})₂, said alkyl, alkenyl, alkynyl or alkoxy groups optionally being substituted with 1, 2 or 3 groups selected from halogen, hydroxy, C₁₋₄ alkoxy, C₁₋₄ alkylthio, haloC₁₋₄ alkoxy, dihaloC₁₋₄ alkoxy, and trihalo₁₋₄ alkoxy;
n is 0, 1 or 2;
Y is selected from oxygen, methylene, sulphur, SO, SO₂ and —N(R^{a})—;
R³ and R⁴ are independently selected from halogen, cyano, C₁₋₄ alkyl, C₂₋₄ alkenyl, C₂₋₄ alkynyl, haloC₁₋₄ alkyl, dihaloC₁₋₄ alkyl, trihaloC₁₋₄ alkyl, C₁₋₄ alkoxy, haloC₁₋₄ alkoxy, dihaloC₁₋₄ alkoxy, trihaloC₁₋₄ alkoxy, methylthio, halomethylthio, dihalomethylthio and trihalomethylthio;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene, NH(CO)—C₀₋₃ alkylene and C₁₋₃ alkyleneC(O)NH—C₁₋₃ alkylene, said alkylene, alkenylene or alkynylene groups or portions of groups optionally being substituted with 1 or 2 groups selected from hydroxy, mercapto, amino, halogen, C₁₋₃ alkyl, C₁₋₃ alkoxy, phenyl, C₁₋₃ alkyl substituted with phenyl, haloC₁₋₃ alkyl, dihaloC₁₋₃ alkyl, trihaloC₁₋₃ alkyl, haloC₁₋₃ alkoxy, dihaloC₁₋₃ alkoxy, trihaloC₁₋₃ alkoxy, and phenyl substituted with 1, 2 or 3 halogen atoms;
R^{c} is selected from hydrogen, C₁₋₄ alkyl, C₂₋₄ alkenyl, C₂₋₄ alkynyl, C₁₋₄ alkoxy, haloC₁₋₄ alkyl, dihaloC₁₋₄ alkyl, trihaloC₁₋₄ alkyl, haloC₁₋₄ alkoxy, dihaloC₁₋₄ alkoxy, and trihaloC₁₋₄ alkoxy;
R⁵ is selected from CO₂R^{d}, PO(OR^{d})₂, —PO(OR^{c})NH₂, —SO₂OR^{d}, —COCO₂R^{d}, CONR^{d}OR^{d}, —SO₂NHR^{d}, —NHSO₂R^{d}, —CONHSO₂R^{d}, and —SO₂NHCOR^{d}; and
each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₋₄ alkyl.

2. A compound of formula (Ia) or a pharmaceutically acceptable ester, amide, solvate or salt thereof, including a salt of such an ester or amide, and a solvate of such an ester, amide or salt, 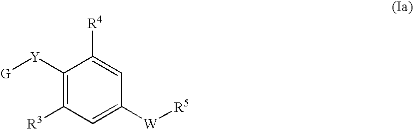
wherein:
G is a group selected from: 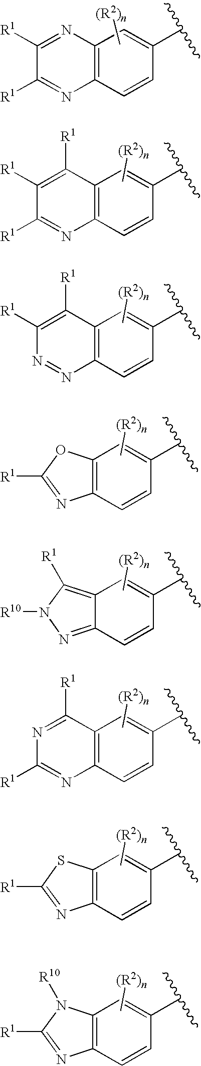
Each R¹⁰ is independently selected from —(CH₂)ₚ—S—R^{b}, —(CH₂)ₚ—SO₂—R^{b}, —(CH₂)ₚ—NH—SO₂—R^{b}, —(CH₂)ₚ—SO₂—NH—R^{b}, —(CH₂)ₚ—NH—CO—R^{b}, —(CH₂)ₚ—CO—NH—R^{b}, C₁₋₈ alkyl, C₃₋₆ cycloalkyl, C₃₋₆cycloalkyl-C₁₋₃ alkyl, phenyl, benzyl and C₃₋₇ heterocyclyl, said alkyl groups or portions of groups optionally being substituted with 1, 2 or 3 groups each independently selected from halogen, hydroxy, N(R^{a})₂, phenyl, haloC₁₋₄alkyl, dihaloC₁₋₄alkyl, trihaloC₁₋₄alkyl, methoxy, halomethoxy, dihalomethoxy, and trihalomethoxy;
p is 1 or 2;
Each R¹ is independently selected from hydrogen, hydroxy, halogen, N(R^{a})₂, —(CH₂)ₘ—S—R^{b}, —(CH₂)ₘ—SO₂—R^{b}, —(CH₂)ₘ—NH—SO₂—R^{b}, —(CH₂)ₘ—SO₂—NH—R^{b}, —(CH₂)ₘ—NH—CO—R^{b}, —(CH₂)ₘ—CO—NH—R^{b}, C₁₋₈ alkyl, C₃₋₆ cycloalkyl, C₃₋₆ cycloalkyl-C₁₋₃ alkyl, phenyl, benzyl and C₃₋₇ heterocyclyl, said alkyl groups or portions of groups optionally being substituted with 1, 2 or 3 groups each independently selected from halogen, hydroxy, N(R^{a})₂, phenyl, haloC₁₋₄alkyl, dihaloC₁₋₄alkyl, trihaloC₁₋₄alkyl, methoxy, halomethoxy, dihalomethoxy, and trihalomethoxy; said cycloalkyl, phenyl or heterocyclyl groups or portions of groups optionally being substituted with 1, 2 or 3 groups independently selected from halogen, hydroxy, C₁₋₄ alkyl, methoxy, halomethoxy, dihalomethoxy, and trihalomethoxy;
m is 0, 1 or 2;
R^{a} is independently selected from a hydrogen atom and a C₁₋₄ alkyl group optionally substituted with 1, 2 or 3 groups independently selected from halogen, methoxy, halomethoxy, dihalomethoxy and trihalomethoxy;
R^{b} is independently selected from hydrogen, C₁₋₄ alkyl, C₂₋₄ alkenyl, C₂₋₄ alkynyl, fluoromethyl, difluoromethyl, or trifluoromethyl, benzyl, heterocyclyl and phenyl, said alkyl, alkenyl, alkynyl or phenyl groups or portions of groups optionally being substituted with 1, 2 or 3 groups independently selected from C₁₋₄ alkyl, halogen, hydroxy, methoxy, halomethoxy, dihalomethoxy and trihalomethoxy;
Each R² is independently selected from halogen, mercapto, C₁₋₄ alkoxy, C₁₋₄ alkyl and N(R^{a})₂, said alkyl or alkoxy groups or portions of groups optionally being substituted with 1, 2 or 3 groups selected from halogen, hydroxy, C₁₋₄ alkylthio, methoxy, halomethoxy, dihalomethoxy, and trihalomethoxy;
n is 0, 1 or 2;
Y is selected from oxygen, methylene, sulphur, SO, SO₂ and —N(R^{a})—;
R³ and R⁴ are independently selected from halogen, C₁₋₄ alkyl, fluoromethyl, difluoromethyl, and trifluoromethyl;
W is selected from C₁₋₃ alkylene, C₂₋₃ alkenylene, C₂₋₃ alkynylene, N(R^{c})—C₁₋₃ alkylene, C(O)—C₁₋₃ alkylene, S—C₁₋₃ alkylene, O—C₁₋₃ alkylene, C₁₋₃ alkylene-O—C₁₋₃ alkylene, C(O)NH—C₁₋₃ alkylene and NH(CO)—C₀₋₃ alkylene, said alkylene, alkenylene or alkynylene groups or portions of groups optionally being substituted with 1 or 2 groups selected from hydroxy, mercapto, amino, halogen, C₁₋₃ alkyl, C₁₋₃ alkoxy, haloC₁₋₃ alkyl, dihaloC₁₋₃ alkyl, trihaloC₁₋₃ alkyl, haloC₁₋₃ alkoxy, dihaloC₁₋₃ alkoxy, and trihaloC₁₋₃ alkoxy;
R^{c} is selected from hydrogen, C₁₋₂ alkyl, fluoromethyl, difluoromethyl, and trifluoromethyl;
R⁵ is selected from CO₂R^{d}, —PO(OR^{d})₂, —SO₂OR^{d}, —NHSO₂R^{d}, —COCO₂R^{d}, and CONR^{d}OR^{d}; and
each R^{d} is independently selected from hydrogen, C₁₋₄ alkyl, C₂₋₄ alkenyl, C₂₋₄ alkynyl, C₃₋₇ heterocyclyl, C₅₋₁₀ aryl and C₅₋₁₀ aryl substituted with 1, 2 or 3 groups independently selected from amino, hydroxy, halogen or C₁₋₄ alkyl.

3. A compound as claimed in claim 1 for use as a medicament.

4. A compound as claimed in claim 3 for use in the treatment or prophylaxis of a condition associated with a disease or disorder associated with thyroid receptor activity.

5. A method for the treatment or prophylaxis of a disease or disorder associated with thyroid receptor activity in a mammal, which comprises administering to the mammal a therapeutically effective amount of a compound of formula (I) as defined in claim 1 or a pharmaceutically acceptable ester, amide, solvate or salt thereof, including a salt of such an ester or amide, and a solvate of such an ester, amide or salt.

6. (canceled)

7. A pharmaceutical composition comprising a compound of formula (I) as defined in claim 1 or a pharmaceutically acceptable ester, amide, solvate or salt thereof, including a salt of such an ester or amide, and including a solvate of such an ester, amide or salt, and a pharmaceutically acceptable excipient.

8. A pharmaceutical composition as claimed in claim 7 further comprising an additional therapeutic agent selected from cholesterol/lipid lowering agents, hypolipidemic agents, anti-atherosclerotic agents, anti-diabetic agents, anti-osteoporosis agents, anti-obesity agents, growth promoting agents, anti-inflammatory agents, anti-anxiety agents, anti-depressants, anti-hypertensive agents, cardiac glycosides, appetite suppressants, bone resorption inhibitors, thyroid mimetics, anabolic agents, anti-tumor agents and retinoids.

9. A method for the diagnosis of conditions associated with a disease or disorder associated with thyroid receptor activity comprising administering a compound as defined in claim 1 in labelled form as a diagnostic agent.

10. A method for identifying ligands for the thyroid hormone receptor comprising utilizing a compound as defined in claim 1 or a labelled form of such a compound as a reference compound.

11. A compound as claimed in claim 4, wherein the condition associated with a disease or disorder associated with thyroid receptor activity is selected from (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes; (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

12. A method for preparing a compound of formula (I) as defined in claim 1 wherein Y is selected from oxygen, sulphur, SO, SO₂ and —N(R^{a})—,
comprising a step of reacting
a compound of formula (II) 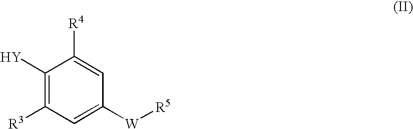
wherein W, R³, R⁴, and R⁵ are as defined in claim 1 and Y is selected from oxygen, sulphur, and —N(R^{a})—
with a compound of formula (III) 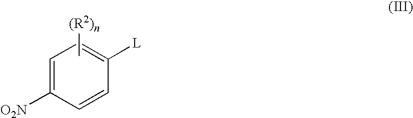
wherein R² is as defined in claim 1 and L is a suitable leaving group, optionally in the presence of a suitable base and, optionally, in the presence of copper powder, followed by reduction of the nitro group to an amino group using a suitable reducing agent, followed by interconversion to a compound of formula (I) as defined in claim 1.

13. A method for preparing a compound of formula (I) as defined in claim 1 wherein G is the following group: 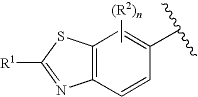
comprising a step of reacting
a compound of formula (IV) 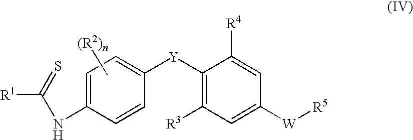
wherein W, Y, R¹, R², R³, R⁴, and R⁵ are as defined in claim 1
with a suitable oxidising agent in the presence of a suitable base, followed optionally by interconversion to another compound of formula (I) as defined in claim 1.

14. A method for preparing a compound of formula (I) as defined in claim 1 wherein G is the following group: 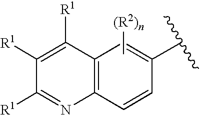
comprising a step of reacting
a compound of formula (V) 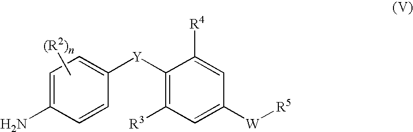
wherein R², R³, R⁴, R⁵, Y and W are as defined in claim 1
with a a compound of formula (VI) 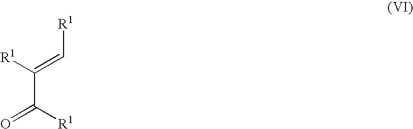
wherein R¹ is as defined in claim 1
in the presence of a suitable acid, followed optionally by interconversion to another compound of formula (I) as defined in claim 1.

15. A method for preparing a compound of formula (I) as defined in claim 1 wherein G is the following group: 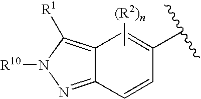
comprising a step of reacting
a compound of formula (VII) 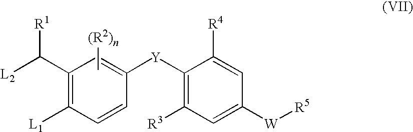
wherein R¹, R², R³, R⁴, R⁵, Y and W are as defined in claim 1 and L₁ and L₂ are suitable leaving groups;
with a hydrazine compound of formula (VIII) 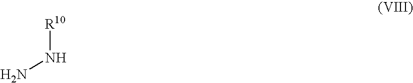
wherein R¹⁰ is as defined in claim 1, followed optionally by interconversion to another compound of formula (I) as defined in claim 1

16. A method for preparing a compound of formula (I) as defined in claim 1 wherein G is the following group: 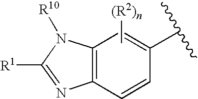
comprising a step of reacting
a compound of formula (IX) 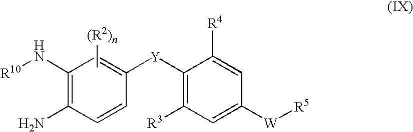
wherein R¹⁰, R², R³, R⁴, R⁵ and W are as defined in claim 1
with a a compound of formula (X) 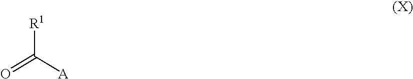
wherein R¹ is as defined in claim 1 and A is H, OH, Cl or OCOR where R is a C₁₋₄ alkyl group
in the presence of a suitable acid, followed optionally by interconversion to another compound of formula (I) as defined in claim 1.

17. A method for preparing a compound of formula (I) as defined in claim 1 wherein G is the following group: 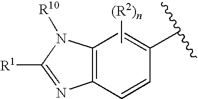
comprising a step of reacting
a compound of formula (XI) 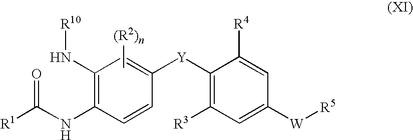
wherein R¹, R¹⁰, R², R³, R⁴, R⁵, Y and W are as defined in claim 1 in the presence of a suitable acid, followed optionally by interconversion to another compound of formula (I) as defined in claim 1.

18. A method for preparing a compound of formula (I) as defined in claim 1 wherein Y is methylene, comprising a step of reacting
a compound of formula (XIII) 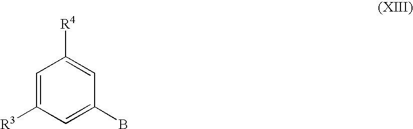
wherein R³ and R⁴, are as defined in claim 1 and B is a group suitable for interconversion to the group —W—R⁵ as defined in claim 1
with a compound of formula (XIV) 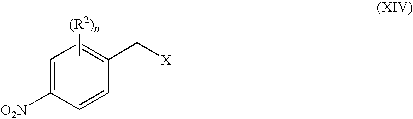
wherein R² is as defined in claim 1 and X is a suitable leaving group, in the presence of a suitable base, followed by conversion of the group B to the group —W—R⁵ as defined in claim 1, and reduction of the nitro group to an amino group using a suitable reducing agent, followed by interconversion to a compound of formula (I) as defined in claim 1.

19. A method for preparing a compound of formula (I) as defined in claim 1 wherein Y is selected from oxygen, sulphur or —N(R^{a})—,
comprising a step of reacting
a compound of formula (II) 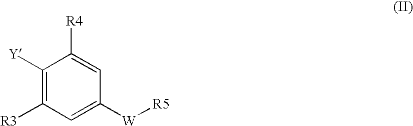
wherein W, R³, R⁴, and R⁵ are as defined in claim 1 and Y′ is OH, SH or NR^{a}H
with a compound of formula (XV)
G-Z (XV)
wherein G is a group selected from: 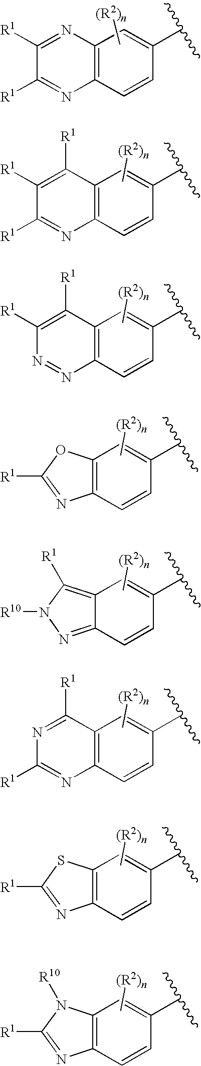
wherein R¹, R¹⁰, R² and n are as defined in claim 1 and Z is a suitable leaving group, optionally in the presence of a suitable base and optionally, in the presence of copper powder, followed optionally by removal of the protecting group, if present, and optionally by interconversion to another compound of formula (I) as defined in claim 1.

20. A method for preparing a compound of formula (I) as defined in claim 1 wherein Y is methylene, comprising a step of reacting
a compound of formula (XVI) 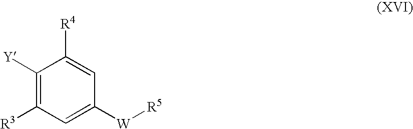
wherein W, R³, R⁴, and R⁵ are as defined in claim 1 and Y′ is CHO
with a compound of formula (XVII)
G-Z (XVII)
wherein G is a group selected from: 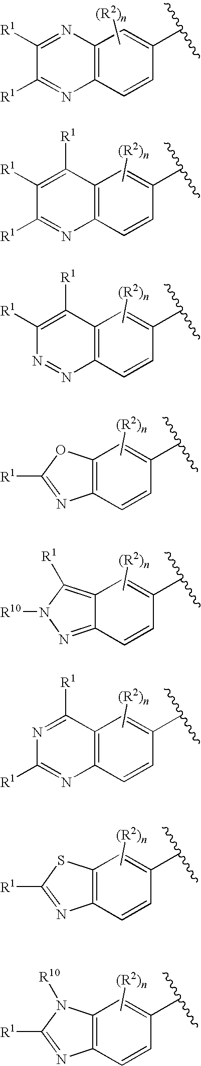
and wherein R¹, R¹⁰, R² and n are as defined in claim 1 and Z is lithium, a Mg-halide, such as MgBr or MgCl, or a derivative of Sn, Pd, B or Cu.
